(19) United States
(12) Patent Application Publication
(10) Pub. No.: US 2008/0248024 A1
(43) Pub. Date: Oct. 9, 2008

(54) THERAPEUTIC AGENT FOR CANCER, INFLAMMATION, AND AUTO-IMMUNE DISEASE CONTAINING INHIBITOR OF ZINC FINGER PROTEIN 91

(75) Inventors: Jung Joon Lee, Daejeon (KR); Jeong-Hyung Lee, Daejeon (KR); Kyeong Lee, Daejeon (KR); Young-Soo Hong, Daejeon (KR); Xuejun Jin, Daejeon (KR)

Correspondence Address: KLARQUIST SPARKMAN, LLP, 121 SW SALMON STREET, SUITE 1600, PORTLAND, OR, 97204, US

(73) Assignee: Korea Research Institute of Bioscience and Biotechnology

(21) Appl. No.: 12/039,640

(22) Filed: Feb. 28, 2008

(30) Foreign Application Priority Data
Feb. 28, 2007 (KR) 10-2007-0020561

(51) Int. Cl. A61K 39/395; A61P 35/00; A61K 31/70; G01N 33/53; A61P 9/00; A61P 29/00; C12Q 1/68; A61K 38/02

(52) U.S. Cl. 424/130.1; 514/44; 514/2; 435/6; 435/7.1

(57) ABSTRACT

The present invention relates to a use of ZFP91 based on the functions of ZFP91 (Zinc Finger Protein 91) and the interaction of ZFP91 with NF-κB (Nuclear factor kappa B) signal transduction pathway proteins, more precisely a method to inhibit the activation of NF-κB alternative pathway by regulating ZFP91 activation, to inhibit tumor growth by inhibiting the transcription factor HIF-1 (hypoxia inducible factor-1) activation, to inhibit cancer malignancy by inhibiting angiogenesis, or reversely a method to increase the activation of NF-κB alternative pathway or to increase angiogenesis by increasing activation of HIF-1. The method of regulating ZFP91 activation of the present invention can increase or reduce HIF-1α stability by increasing or reducing the activation of NF-κB alternative pathway, so that it can be effectively used for the development of an anticancer agent, a therapeutic agent for arthritis, a therapeutic agent for ulcerative colitis, an anti-inflammatory agent and an angiogenesis inducer.

This application claims priority to Korea patent Application Number 2007-20561, filed Feb. 28, 2007, the specification of which is incorporated by reference in its entirety.

### TECHNICAL FIELD

The present invention relates to a use of ZFP91 (Zinc Finger Protein 91) based on the function of ZFP91 and its interaction with proteins on signal transduction pathway of the transcription factor NF-κB (Nuclear factor kappa B). More precisely the present invention relates to a method to inhibit tumor cell proliferation by suppressing the activity of NF-κB alternative pathway and the activity of transcription factor HIF-1 (hypoxia inducible factor-1) by regulating the activity of ZFP91; to inhibit malignancy of cancer by suppressing angiogenesis; to regulate chronic inflammatory disease caused by over-activation of alternative pathway such as arthritis, inflammatory colitis, multiple sclerosis, chronic hepatitis, etc, and lymphoma; and reversely, to increase angiogenesis by promoting the activity of NF-κB alternative pathway or the activity of HIF-1.

### BACKGROUND ART

Tumorigenesis, the development process of cancer from a normal cell, is composed of the following stages: the early tumor stage where gene is mutated: the tumor progressing stage where cell proliferation increases but apoptosis decreases: the malignant tumor stage where cancer cell invasion and metastasis occur and thereby tumor cells move to other tissues and are growing there. This process is stimulated by inflammation and auto-immune response. Particularly, inflammation plays an important role in tumorigenesis. It has been reported that pro-inflammatory cytokines such as Interleukin (IL-1), Interleukin-6 (IL-6), and tumor necrosis factor-α(TNF-α) or chemokines such as CXC-chemokine ligand 8 (CXCL8) play an important role in stimulating tumor growth and malignancy.

Cytokines or chemokines are the genes whose expressions are regulated by the transcription factor NF-κB (Nuclear factor kappa B). Reversely, they can activate NF-κB. The activated NF-κB regulates the expressions of such genes involved in cell proliferation, apoptosis, angiogenesis and metastasis, and thereby has an effect of accelerating tumorigenesis and progress (M Karin & FR Greten, Nat Rev Immunology 5, 749-759, 2005). It has also been reported that the activity of NF-κB in breast cancer, prostatic cancer, colorectal cancer, uterine cancer, leukemia or lymphoma increases as tumor progresses to malignancy (L T Amundadottir and P Leder, Oncogene 16, 737-756, 1998; Nakshatri et el., Mol Cell Biol 7,3629-3639, 1997). Thus, NF-κB is not only an important factor causing immune inflammatory response but also an important factor connecting chronic inflammation and tumorigenesis, suggesting that NF-κB is an important transcription factor involved in malignancy of cancer (Karin et al., Nat Rev Cancer 2,301-310, 2002).

NF-κB in mammals is composed of 5 subunits such as p65 (RelA), RelB, c-Rel, NF-κB1 (p50) and NF-κB2 (p52). These subunits form a homologous or a heterologous complex, which exists in cytoplasm with being combined with IκB family, the inhibitor proteins, before being activated. When IκB is phosphorylated by IKC (I kappa B kinase complex), which means IκB is degraded by ubiquitination, NF-κB is released, activated and moves into nucleus and is functioning there. P105 and p100, the precursors of NF-κB1 and NF-κB2, are combined with other NF-κB proteins to inhibit their activation. They contain IκB homologous inhibition region at C-terminal. So, dimers binding to these proteins can only exist in cytoplasm owing to the function of IκB homologous inhibition region. But once a stimulus is given to a cell, the IκB homologous inhibition regions of p105 and p100 are degraded and thereby active p50 and p52 are generated to form a homo or hetero complex that can move into the nucleus (S Gosh and M Karin, Cell 109, 81-96, 2002).

NF-κB activation pathway is largely divided into classical pathway and alternative pathway. These pathways are differently regulated according to the kind of kinase (Viatour et al., Trends Biochem Sci 1, 43-52, 2005). The best known classical pathway which has been the best target of study is activated by pro-inflammatory cytokines such as TNF-α or IL-1β and the representative subunit of NF-κB activated by such classical pathway is p65(RelA)/p50. This pathway attracts adaptor proteins such as TNF-receptor-associated death domain protein (TRADD), receptor interacting protein (RIP) and TNF-receptor-associated factor2 (TRAF2) and is combined with those proteins on intracellular receptor membrane when a stimulus is given from outside (Hsu et al., Cell 81, 495-504, 1995). Accordingly, IKK complex becomes activated. Particularly this signal pathway depends on IKKβ. IKKβ forms IKK complex together with IKKα, another catalytic subunit, and IKKγ (NEMO), a regulatory subunit.

IKKβ induces phosphorylation of IκB, leading to ubiquitination of IκB. It degrades IκB by proteasome and then NF-κB dimer bound to IκB is separated from IκB and moves into nucleus to induce the expressions of specific NF-κB target genes. The alternative pathway activated by lymphotoxin β (Dejardin et al., Immunity 17,525-535, 2002) B-cell activating factor (BAFF) (Claudio et al., Nat Immunol 3, 958-965, 2002) or CD40 ligand (Coope et al., EMBO J 21, 5375-5385, 2002) and Epstein-Barr virus (Xiao et al., Mol Cell 7, 401-409, 2001) and human T-cell leukemia virus (Elipous et al., Oncogene 22, 7557-7569, 2003; Solan et al., J Biol Chem 277, 1405-1418, 2002; Kayagaki et al., Immunity 17, 515-524, 2002; Hatada et al., J Immunol 171, 761-768, 2003) depends on NF-κB-inducing kinase (NIK/MAP3K14) and IKKα. When p100 is degraded, RelB/p52 dimer is formed, which moves into the nucleus with carrying this pathway. When a stimulus is given to a cell, TNF receptor associated factor (TRAF) protein and NIK are combined in receptor to activate NIK and induce phosphorylation of IKKα homodimer. Then, p100 is phosphorylated. As a result, IκB homologous inhibition region of p100 is degraded to generate p52. The generated p52 binds to RelB protein, which moves into the nucleus (Senftleben et al., Science 293, 1495-1499, 2001; Solan et al., J Biol Chem 277, 1405-1418, 2002; Kayagaki et al., Immunity 17, 515-524, 2002; Hatada et al., J Immunol 171, 761-768, 2003). To activate NF-κB, the above two pathways require phosphorylation of a suppressor protein.

In both pathways of NF-κB, when a signal is transmitted, TRAF family is combined on a membrane receptor. TRAF family is one of conjugated protein families that binds to various surface receptors and is capable of activating NF-κB and MAP kinase (mitogen activate protein kinase) along with TNF-α and IL-1β super family. TRAF directly induces cell survival by interacting with various proteins regulating apoptosis or cell survival or regulates apoptosis caused by death receptor (N K Lee and S Y Lee, J Biochem Mol Biol 35, 61-66, 2002; Z P Xia and Z J Chen Science's STKE www.stke.org, 1-3, 2005). The receptor oligomer induced by a ligand attracts a member of TRAF family to the receptor. As a result, a signal complex comprising various proteins is generated, which will activate kinase cascade stimulating the activation of NF-κB or activation protein 1 (AP-1), and protein kinase B (Akt/PKB) (Means et al., Cyt Growth Factor 11, 219-232, 2000).

Among the members of TRAF family, TRAF2 was found first and studied most. TRAF2 was confirmed by numbers of experiments to be an important factor composing TNF-α receptor super family signal transduction pathway (Song et al., Proc Natl Acad Sci 94, 9792-9796, 1997). It has also been confirmed by in vivo gene-targeted deletion and over-expression of dominant negative form of TRAF2 that TRAF2 provides anti-apoptosis signals (Lee and Lee, J Biochem Mol Biol 35, 61-66, 2002).

NIK was identified as a MAP3K kinase interacting with TRAF2 at first. According to the previous report, NIK directly interacts with TRAF2, IKKα and IKKβ, and is capable of activating NF-κB by stimulus of TNF/NGF receptor family (Malinin et al., Nature 385, 540-544, 1997). In previous studies using NIK mutation, it seemed that NIK played an important role in TNF-α mediated NF-κB activation. But later, studies using NIK knock-out mice reported that NIK was an essential factor for IKKα mediated p100 phosphorylation and IκB homologous inhibition region digestion, which is essential for the activation of NF-κB alternative pathway. According to recent reports, NIK possibly activates both classic pathway and alternative pathway of NF-κB in the presence of specific inducers (Ramakarishnan et al., Immunity 21, 477-489, 2004), but over-activation of alternative pathway plays a certain role in the development of chronic inflammatory diseases such as rheumatoid arthritis, inflammatory colitis, multiple sclerosis, chronic hepatitis, and B cell lymphoma (Dejardin E, Biochem Pharmacol 72, 1161-1179, 2006).

NF-κB activated by the above pathways regulates the expressions of specific genes including Cyclin D1 or c-Myc involved in cell proliferation, cIAPs, BCL-X_{L} and c-FLIP inducing anti-apoptosis, various chemokines or cytokines involved in immunity and inflammation and adhesion molecules. So, NF-κB is closely related to cancer and inflammatory response (Karin et al., Nat Rev Cancer 2, 301-310, 2002) and NF-κB inhibitor has been used as an anti-inflammatory agent and an anticancer agent and is still an important target of study (M Karin et al., Nature Rev. Drug Discovery 3, 17-26, 2004; Nakanishi C & Toi M, Nature Rev. Cancer 5, 297-309, 2005).

NF-κB stabilizes the transcription factor hypoxia inducible factor-1 alpha (HIF-1α) playing an important role in cancer malignancy and metastasis and increasing the expression of the angiogenesis related factor VEGF (Jung et al., FASEB J, Express article 10.1096/fj.03-0329fje. published online Sep. 4, 2003; Zhou et al., Cancer Res 64, 9041-9048, 2004). Hypoxia is generally observed in cancer, particularly in solid cancer. Solid cancer cells have been adapted to the hypoxic condition through various genetic modifications, so that cancer cells become more malignant and have resistance against an anticancer agent. In fact, hypoxia is known as a major factor aggravating tumor in at least 70% of all the human carcinoma (Nature 386, 403, 1997; Hockel M and Vaupel P, Semin. Oncol. 28, 36-41, 2001; Nature Med. 6, 1335, 2000; Bos et al. Cancer 2003, 97, 1573-1581). HIF-1 is the most important molecule in regulation of cancer cells under hypoxic condition. HIF-1α protein level is closely related to the prognosis of a cancer patient.

HIF-1 (Hypoxia Inducible Factor-1) is a transcription factor induced in hypoxia, which is a heterodimer composed of HIF-1α subunit degraded oxygen-dependently and HIF-1β subunit being expressed constitutively (Cancer Metastasis Rev., 17, 187-195, 1998; Trends Mol. Med., 7, 345-350, 2001).

Under normoxic condition, HIF-1α protein binds to a tumor suppressor gene pVHL (Von Hippel-Lindau) oxygen dose-dependently when proline residue at #402 and #564 is hydroxylated. Then, VHL protein forms a multiple complex with Elongin B and C, Rbx1 and Cullin 2. This complex has E3 ubiquitin ligase activity, so that HIF-1α and its homologous series HIF-2α become ubiquitinated and degraded by proteasome (Nat Rev Cancer 2, 673-682, 2002; Curr Opi Gen Dev 13, 56-60, 2003; Trends Mol Med 10, 146-149, 2004; Trends Mol Med 10, 466-472, 2004). Under hypoxic condition, the above reaction is suppressed, so that HIF-1α protein is accumulated and binds to HIF-1β protein which has been there already to be functioning as a transcription factor in nucleus (Science 292, 468-472, 2001; Science 292, 468-472, 2001). The stability of HIF-1α is affected by factors involved in oxygen sensing pathway, in addition to oxygen partial pressure. These factors are exemplified by transition metal ion, iron chelator and antioxidant, etc. HIF-1α protein is also accumulated by the activation of growth factors such as epidermal growth factor, heregulin, insulin-like growth factor-I and insulin-like growth factor-II or oncogene such as ErbB2. When these growth factors are conjugated to each corresponding receptor, PI3K-AKT and MAPK signal transduction pathways are activated, so that HIF-1α protein synthesis is increased and thus HIF-1α protein is accumulated.

HIF-1 moved into nucleus is bound to HRE (Hypoxia Responsive Element, 5′-ACGTG-3′) on the promoter of a target gene to induce the expression of the same. Up to date, approximately at least 60 kinds of genes that can be regulated by HIF-1 including VEGF (vascular endothelial growth factor) have been identified (Nat. Rev. Cancer 2, 38-47, 2002; J. Biol. Chem. 278, 19575-19578, 2003; Nature Med. 9, 677-684, 2003; Biochem. Pharmacol. 64, 993-998, 2002).

The representative genes whose expressions are regulated by HIF-1 are hexokinase 2, glucose transporter 1, erythropoietin, IGF-2, endoglin, VEGF, cMet MMP-2, uPAR, MDR1, etc. When these genes are over-expressed, cancer cells acquire resistance against apoptosis, capability of angiogenesis and cell proliferation, capability of invasion and resistance against anti-cancer agents, resulting in cancer malignancy and metastasis. As stated, HIF-1 plays an important role in tumor growth, particularly solid tumor growth, proliferation and malignancy, making it as an important target of study to develop a novel anticancer agent (Cancer Res. 62, 4316, 2002; Nature Rev Drug Discovery 2, 1, 2003; Semenza et al. Nature Rev Cancer 3, 721-732, 2003).

VEGF is an important cell growth factor for angiogenesis and regulated by HIF-1 and NF-κB (Nature 359, 843-845, 1992; Nature 359, 845-848, 1992; Tong et al., Respir Res 7, 37, 2006 available from: http://respiratory-research.com/content/7/1/37). Cancer cells cannot be proliferated without oxygen and nutrition supplied through blood vessels. So HIF-VEGF pathway is involved in cancer cell proliferation, metastasis and angiogenesis (PNAS USA 94, 8104-8109, 1997; Can Res 60, 4010-4015, 2000). HIF inhibitors and VEGF pathway blockers have been major targets of the development of a novel anticancer agent (Opthalmology 109, 1745-1751, 2002), and some of them are already commercialized (ex. Avastin) (Proc Am Soc Clin Oncol 21, 15, 2002).

HIF-1 is not only useful for the treatment of cancer but also useful as a therapeutic agent candidate for the treatment of such disease that is progressed by the activation of angiogenesis. Angiogenesis factor such as VEGF activated by HIF-1 under hypoxic condition is related to the progress of not only cancer but also diabetic retinopathy and arthritis. Therefore, a compound that is capable of suppressing HIF-1 activated under hypoxic condition in a disease tissue can be used as a novel therapeutic agent for the treatment of diabetic retinopathy or rheumatoid arthritis (Eiji Ikeda, Pathology International, 2005, Vol 55, 603-610).

In the parallel with the development of a novel anticancer agent using HIF-1 or VEGF inhibitor, studies to treat vascular diseases such as ischemic diseases by increasing the expression or activity of HIF-1 or VEGF have been actively undergoing. Ischemic disease includes cardiovascular disease caused by the interruption of blood flow, which is exemplified by myocardial ischemia and peripheral vascular disease. VEGF which has been used for the treatment of ischemic diseases (Yla-Herttuala S and Alitalo K. Nat. Med. 9(6):694-701, 2003; Khan T A et al., Gene Ther. 10(4):285-91, 2003) has been confirmed to induce angiogenesis in animal models (Leung D W et al., Science. 8; 246(4935):1306-9, 1989; Dvorak H F et al., Am J Pathol. 146(5):1029-39, 1995). Also, the effect of adenovirus gene delivery system inserted with VEGF (Ad.VEGF) was investigated in ischemic myocardium and muscle cell models. As a result, blood vessels increased significantly (Mkinen K et al., Mol. Ther. 6, 127-133, 22002). The blood vessels newly generated for 4 weeks during which the expression of VEGF was induced in animal models did not disappear even after the expression of VEGF was not induced any more and the functions of tissues were rather improved (Dor Y et al., EMBO J. 21, 1939-1947, 2002). Gene therapy for coronary artery occlusion syndrome and peripheral insufficiency using VEGF inserted adenovirus vector is clinically tested (Maekimen K et al., Mol Ther 6, 127-133, 2002; Stewart D J et al. Circulation 106, 23-26, 2002; Rajagopalan S et al., J Am Coll Cardil 41, 1604, 2003). Gene therapy for myocardial ischemia using HIF-1 introduced adenovirus vector is in phase 1 of clinical test (Vincent K A et al., Circulation 102, 2255-2261, 2000). The clinical tests using HIF-1α and VEGF for gene therapy for ischemic diseases have been taken but angiogenesis related experiment to investigate the promotion of VEGF expression by stabilizing HIF-1α has not been reported yet.

Kamebakaurin (KA) is a kaurane-type diterpenoid compound isolated from Isodon japonicus Hara which has been used for the treatment of chronic gastritis and cancer in traditional medicine. This compound inhibits the activity of NF-kB by suppressing DNA binding activity of p50 subunit of NF-κB. This compound has excellent anti-inflammatory effect in an air-pouch model and an arthritis model (Hwang et al., Planta Medica 67, 406-410, 2001, Lee et al., J Biol Chem 277, 18411-18420, 2002, Planta Medica 70,526-530, 2004; U.S. Pat. No. 6,894,073 B2, May 17, 2005). ZFP91 is known as a human analogue of mouse zinc finger protein 19. ZFP91 is allegedly over-expressed in acute leukemic cells (Unoki et al., Int J Oncol 22, 1217-1223, 2003). Recently, it was reported that ZFP91 is conjugated to ARF (alternative reading frame protein), known as a tumor suppressor protein (Tompkins et al., Cell Cycle 5, 641-646, 2006). However, its functions and role in cancer cells are not disclosed yet.

### DISCLOSURE

### Technical Problem

It is an object of the present invention to provide a method for inhibiting tumor growth and metastasis by examining interrelationship between the intracellular functions of ZFP91, whose expression is regulated by NF-κB activation and which has oncogene activity, and tumorigenesis, progression, metastasis and angiogenesis, blocking NF-kB pathway by suppressing ZFP91 activity and inhibiting stabilization and activation of HIF-1α. It is another object of the present invention to provide a method for using a ZFP1 inhibitor for the treatment of angiogenesis related diseases such as diabetic retinopathy and arthritis caused by HIF-1 mediated up-regulation of VEGF under hypoxic condition. ZFP91 activates alternative pathway of NF-κB so that it can be used for the development of a therapeutic agent for chronic inflammatory diseases such as rheumatoid arthritis, inflammatory colitis, multiple sclerosis, chronic hepatitis, etc, and B cell lymphoma (Dejardin E, Biochem Pharmacol 72, 1161-1179, 2006). It is also an object of the present invention to provide a method for inducing cell proliferation, for inhibiting apoptosis, and for treating ischemic diseases by increasing the expression of an angiogenesis factor VEGF by increasing the activation of ZFP91 to bring the activation of NF-κB pathway and HIF-1α stabilization.

### Technical Solution

The present inventors identified ZFP91 regulated by KA by cDNA microarray (FIG. 1 and FIG. 2) and found out two NF-κB binding transcription regulation sites in its promoter region. And the inventors further confirmed by electrophoretic mobility shift assay that NF-κB was conjugated to the regulatory sites (FIG. 3 and FIG. 4). The present inventors also constructed ZFP91 promoter and its partial mutant (FIG. 3C) and cloned them into pGL3basic vector (Strategene) to construct the plasmid vector (pGL3-ZFP91prom-LUC) for reporter assay. The present inventors confirmed that ZFP91 was the target gene of NF-κB by investigating the expression of the reporter gene induced by various molecules activating NF-κB (FIG. 5) and that various molecules activating NF-κB increased the expression of ZFP91 at mRNA and protein levels. Accordingly, it was confirmed that ZFP91 was the protein whose expression was regulated by NF-κB activation (FIG. 6). From the experiments using pGL3-ZFP91prom-LUC, it was also confirmed that HIF-1α inducing materials such as DFO and CoCl₂ increased the expression of ZFP91 reporter gene (FIG. 5-E). Previous arts have confirmed that ZFP91 is up-regulated in various blood cancers. Based on that, the present inventors examined the level of ZFP91 in other cancers including stomach cancer and breast cancer cell lines and confirmed that ZFP91 was over-expressed therein (FIG. 7). ZFP91 mRNA expression in stomach cancer tissues was measured by in situ hybridization (FIG. 8-10). To investigate the functions of ZFP91, the stomach cancer cell line AGS (ZFP91+) and the breast cancer cell line MCF-7 (ZFP91+) expressing ZFP91 stably were constructed (FIG. 11).

It is well known that NF-κB regulates the expressions of numbers of proteins involved in cancer malignancy and metastasis. Since ZFP91 expression is regulated by NF-κB, ZFP91 has been tested to see if it induces changes of cancer cell characteristics and if it has any effect on the expressions of proteins inhibiting apoptosis or regulating cell cycle. First, to investigate interrelationship between ZFP91 and anchorage independent cell proliferation and cell migration, effect of ZFP91 on colony formation and invasion of cancer cells was investigated. ZFP91 remarkably increased colony forming capacity of the stomach cancer cell line AGS on softagar (FIG. 12). In the experiment using Modified Boyden Chamber method (Cos S et al., Cancer Res 58, 4383-4390, 1998, Koo et al., Oncogene 21. 4080-4088, 2002), the cells in which ZFP91 was over-expressed stably exhibited increased invasion capacity, precisely ZFP91 increased invasion capacity of MCF-7 cell line by 2.1 fold and AGS cell line by 4.6 fold, compared with the control (FIG. 13).

Next, ZFP91 was examined to investigate whether or not it increased the expressions of proteins inhibiting apoptosis or it reduced the expressions of proteins regulating cell cycle. As a result, ZFP91 increased cIAP1 and cIAP2, the most representative apoptosis inhibitor proteins and NF-κB target proteins, but inhibited the expressions of p27 (KIP1) and p21 (CIP1/WAF1) (Sherr C J Science, 274, 1672-1677, 1996), the inhibitors of cyclin dependent kinase (CDK) playing an important role in entering S phase that is a very important stage in cell proliferation, after G1 stage in cell cycle (FIG. 14). In particular, p27 (KIP1) is the protein inducing ubiquitination dependent degradation via CDK2 phosphorylation and regulated by IKKα mediated NF-κB alternative pathway (Schnieider et al., EMBO J. 25, 3801-3812, 2006). The effect of ZFP91 on the expressions of p27 (KIP1) and p21 (CIP1) were investigated in the stomach cancer cell line AGS by Western blotting. As a result, the expressions of p27 and p21 were inhibited in the cells over-expressing ZFP91 (FIG. 14).

To examine the functions of ZFp91 more precisely, a domain which was different from the domain common in Zinc Finger proteins expressed largely in human was expressed and used in a mouse to produce anti-ZFP91 antibody (FIG. 15). ZFP91 siRNA was also constructed to investigate the effect of ZFP91 on apoptosis. MTT assay was performed with MCF-7 cells transformed with control siRNA oligomer or ZFP91 siRNA oligomer. As a result, it was confirmed that ZFP91 siRNA induced apoptosis. AGS cells were transformed with control siRNA and ZFP91 siRNA. As a result, ZFP91 expression was reduced by siRNA. From the observation of morphology of the cells, it was confirmed that the inhibition of ZFP91 expression resulted in apoptosis (FIG. 16). Mouse xenograft models were prepared using MKN-45 cells transformed with control plasmid and ZFP91 expressing plasmid for further experiment. As a result, it was confirmed that ZFP91 accelerated tumor growth and angiogenesis and increased blood VEGF level, proving that ZFP91 is functioning like oncogene promoting tumor growth and metastasis (FIG. 17).

It was also investigated whether or not ZFP91 activated NF-κB pathways, based on the founding that ZFP91 increases the expression of cIAP, the target protein of NF-κB (FIG. 14-A). ZFP91 increased NF-κB dependent reporter gene expression dose-dependently, which was improved by TNF-α (FIG. 18). When cells were transformed with the plasmid GAL4-DBD-p65^{aa268-552} (Lee et al. Biochem Pharmacol 66, 1925-1933, 2003) constructed to measure the NF-κB transcription promoting activity, p65 (RelA) transcription was increased ZFP91 expression plasmid dose-dependently. In the meantime, in Gal-4-DBD-p65 mutant transformed with GAL4-DBD-p65^{aa521-552} plasmid containing only p65 transcription activation domain TA1, p65 (RelA) transcription was significantly increased by ZFP91 expression plasmid dose-dependently. In MCF-7 cells, it was also confirmed that phosphorylation of p65 (RelA) Ser536 was increased by ZFP91 expression plasmid dose-dependently (FIG. 19). ZFP91 is up-regulated by various molecules activating NF-κB (FIG. 5 and FIG. 6). Particularly, when NF-κB is activated by NIK, ZFP91 up-regulation is the greatest. When ZFP91 was co-expressed with NIK dominant negative form, ZFP91 mediated NF-κB activation was not observed (FIG. 21).

Functions of ZFP91 on NF-κB pathways were investigated. As a result, ZFP91 remarkably increased phosphorylation of p65 protein and p38 MAPK by NIK, the important activator molecule for the activation of NF-κB alternative pathway. More importantly, the in vivo prodiction of p52, the protein playing an important role in NF-κB alternative pathway and activated by NIK and IKKα, was increased ZFP91 dose-dependently. The level of p52 in nucleus was also increased by ZFP91 (FIG. 20). However, ZFP91 did not increase phosphorylation of IKKβ and had nothing to do with IκBA degradation (FIG. 20A). The above results indicate that ZFP91 is functioning in NIK mediated NF-κB alternative pathway.

NIK was identified as a MAP3K kinase interacting with TRAF2. It also interacts directly with TRAF2, IKKα, and IKKβ. According to the previous report, NIK strongly activates NF-κB by stimulating TNF/NGF receptor family (Malinin et al., Nature 385, 540-544, 1997). It was further investigated whether or not ZFP91 could activate NF-κB alternative pathway by combining with NIK and TRAF2. As a result, ZFP91 formed a complex with TRAF2 and NIK (FIG. 22) and Zinc Finger Domain of ZFP91 played an important role in formation of the complex (FIG. 23). ZFP91 was confirmed to form a complex with any fragment that contains kinase domain of NIK (FIG. 24).

In relation to the stabilization and activation of NIK, there is no report on TRAF2's effect on NIK except the report that TRAF3 is involved in degradation of NIF by ubiquitination (Liao et al., J Biol Chem 279, 26243-26250, 2004) and p52/RelB activity increases in TRAF2 (Marin et al., Nature 385, 540-544, 1997) deficient B cells (Grech et al., Immunity 21, 629-642, 2004). Thus, the present inventors investigated whether or not ZFP91 induced poly-ubiquitination of NIK in HEK293 cells. The results confirmed that ZFP91 had the activity of inducing poly-ubiquitination of NIK (FIG. 26). The in vitro ubiquitination was further investigated and the result confirmed that ubiquitination was accelerated in the presence of NIK (FIGS. 26-29), suggesting that ZFP91 has the activity of inducing stabilization and activation of NIK by NIK ubiquitination.

Although the functions of NF-κB such that it stabilizes HIF-1α, the transcription factor playing an important role in cancer malignancy and metastasis, and increases the expression of VEGF, the angiogenesis factor (Jung et al., FASEB J. express article 10.1096/fj.03-0329fje. published online Sep. 4, 2003; Zhou et al., Cancer Res 64, 9041-9048, 2004), the mechanism of such functions has not been disclosed. In this invention, experiment was already performed using the plasmid pGL3-ZFP91prom-LUC constructed by using two NF-kB binding transcription regulatory sites of ZFP91, and as a result, it was disclosed that the molecules inducing hypoxia inducible factor-1 such as DFO (deferoxamine) and CoCl₂ increase ZFP91 expression (FIG. 5-E). Based on that, interaction between ZFP91 and HIF-1α, precisely in activation and expression, was further investigated. The results confirmed that ZFP91 activated and stabilized HIF-1 in AGS cells, HT-29 cells and Hep3B cells dose-dependently regardless of oxygen level, confirmed by hypoxia response element dependent report assay and Western blotting. It was also confirmed by Northern blotting that ZFP91 increased the expression of vascular endothelial growth factor (VEGF) (FIG. 25, FIG. 31, FIG. 32). The above effects were suppressed by ZFP91 siRNA (FIG. 33). ZFP91 increased the expression of HIF-1 target genes regardless of normoxic or hypoxic condition, which was suppressed by ZFP91 siRNA (FIG. 33). It was also investigated whether or not ZFP91 could stabilize HIF-1α playing an important role in cancer malignancy and metastasis. As a result, it was confirmed that ZFP91 formed a complex with pVHL and HIF-1α and increased ubiquitination of HIF-1α (FIG. 34 and FIG. 35). It was further confirmed that ZFP91 formed multiple complex with Elongin B and C, Rbx 1 and Cullin 2, which exhibited E3 ubiquitin ligase activity, so that ZFP91 had the effect of reducing the expression of the tumor suppressor gene pVHL (Von Hippel-Lindau) but increasing the expression of UCP, the protein known to degrade pVHL by ubiquitination (Jung et al., Nature Med 12, 809-816, 2006) (FIG. 35).

The above results indicate that inhibition of the expression or functions of ZFP91 leads to anticancer effect and inhibition of metastasis. Therefore, it was examined whether or not the compounds inhibiting the expression or functions of ZFP91 had anticancer activity. First, it was investigated whether or not kamebakaurin, celastrol and parthenolide which have been known to have anticancer activity as NF-κB inhibitors could inhibit the expressions of ZFP91 and HIF-1α. In AGS cells expressing FLAG-ZFP91, the NF-κB inhibitors kamebakaurin (KA), celastrol (cel) and parthenolide (PTN) inhibited TNF-α mediated ZFP91 expression and inhibited HIF-1α expression induced under 1% partial oxygen pressure (FIG. 37). Kamebakaurin demonstrated anticancer activity inhibiting cell growth of the malignant breast cancer cell line MDA-MB-435 and metastasis to lung in the xenograft model (FIG. 38). The compounds inhibiting ZFP91 inhibit the expression of HIF-1α, so that they can be leading compounds for the development of a novel anticancer agent (FIG. 39).

In conclusion, ZFP91 expression is regulated by NF-κB and seems to have auto regulation mechanism increasing the transcriptional activity of NF-κB in return. By regulating the expressions of NF-κB target genes, ZFP91 activates NF-κB alternative pathway which is important for cell proliferation and is also functioning to increase the expression of VEGF, the representative target gene, by stabilizing and activating HIF-1α. So, ZFP91 has been proved to be a very important protein involved in tumor growth and malignancy including metastasis.

Hereinafter, the present invention is described in detail.

To achieve the above objects, the present invention provides a method for inhibiting cancer containing the step of administering the pharmaceutically effective dose of ZFP91 (Zinc finger protein 91) inhibitor to a subject with cancer.

The present invention also provides a method for reducing NF-κB activity and HIF-1α stability, and inhibiting VEGF expression by suppressing ZFP91 (Zinc finger protein 91) activity.

The present invention also provides a method for screening a regulator of expression or activity of ZFP91.

The present invention further provides an anticancer agent containing a ZFP91 inhibitor as an active ingredient.

The present invention also provides a therapeutic agent for the treatment of as diabetic retinopathy and chronic inflammatory disease such as rheumatoid arthritis, inflammatory colitis, multiple sclerosis and chronic hepatitis, containing a ZFP91 inhibitor as an active ingredient (Eiji Ikeda, Pathology International, 2005, Vol 55, 603-610, Dejardin E, Biochem Pharmacol 72, 1161-1179, 2006).

The present invention also provides a method for screening a ZFP91 and NIK binding inhibitor.

The present invention provides a method for increasing HIF-1α stability and promoting VEGF expression by increasing ZFP91 activity.

The present invention provides a VEGF expression promoter containing a ZFP91 activity enhancer, an expression vector containing ZFP91 gene or ZFP91 protein as an active ingredient.

The present invention provides an angiogenesis promoter containing a ZFP91 activity enhancer, ZFP91 gene, an expression vector containing ZFP91 gene or ZFP91 protein as an active ingredient.

The present invention provides a method for diagnosing cancer and its prognosis by measuring the expression of ZFP91 with diagnostic samples obtained from a patient and a diagnostic kit using the same.

The present invention also provides a method for inducing cell proliferation or inhibiting apoptosis by inducing the expression of ZFP91.

In addition, the present invention provides an EPO production enhancer containing an expression vector containing ZFP91 gene.

Hereinafter, the present invention is described in more detail.

1. The present invention provides a method for inhibiting the activation of NF-κB alternative pathway and an anticancer agent containing a ZFP91 (Zinc Finger Protein 91) inhibitor as an active ingredient.

The present invention also provides a method for inhibiting the activation of HIF-1α and an anti-cancer agent containing a ZFP91 (Zinc Finger Protein 91) inhibitor as an active ingredient.

The present invention also provides a method for inhibiting cancer containing the step of administering the pharmaceutically effective dose of ZFP91 (Zinc finger protein 91) inhibitor to a subject with cancer.

The present inventors identified NF-κB target gene by using kamebakaurin (KA) (Hwang et al., Planta Medica 67, 406-410, 2001; Lee et al., J Biol Chem 277, 18411-18420, 2002; Planta Medica 70,526-530, 2004; U.S. Pat. No. 6,894,073 B2, May 17, 2005) which is the compound inhibiting the activation of NF-κB by inhibiting DNA binding capacity of NF-κB p50. That is, cDNA microarray was performed to screen a gene whose expression was significantly changed by KA. As a result, a gene whose expression was changed at least two fold was detected and the functions of ZFP91 gene were investigated.

ZFP91 is a protein composed of 570 amino acids, which was presumed to be 63 kDa in size but was expressed as 91 kD sized protein in cells and had 5 Zinc finger domains, a coiled coil, leucine zipper pattern and 4 nuclear localization sequences (Unoki et al., Int J Oncol 22, 1217-1223, 2003). The present inventors found out that ZFP91 has two NF-κB binding consensus sequences in −1105 and −1664 regions of 5′ upstream, suggesting that ZFP91 is NF-kB dependent gene (see FIG. 3 and FIG. 4). These sequences were linked to luciferase gene to construct pGL-ZFPprom-LUC.

The present inventors examined whether or not ZFP91 expression was induced by NF-κB when stimulated by TNF-α, PMA and LPS activating NF-κB. As a result, the expression was significantly increased time dependently at protein level, while slightly increased at mRNA level. P65, IKKα, IKKβ, and NIK, which activate NF-κB, were expressed in MCF-7 cells. As a result, ZFP91 mRNA expression was not greatly increased but ZFP91 protein expression was significantly increased. Reporter gene expression induced by pGL-ZFPprom-LUC plasmid was also increased by DFO (deferoxamine) and CoCl₂, both of which induce HIF-1α expression (see FIG. 6, FIG. 5-E). ZFP91 expressions in various cancer cell lines including breast cancer cell lines and stomach cancer cell lines were compared. As a result, ZFP91 over-expression was significantly increased in malignant breast cancer cell line and stomach cancer cell line where NF-κB activity was high (see FIG. 7). ZFP91 mRNA expression was investigated by in situ hybridization. As a result, ZFP91 expression was increased rather in cancer tissues (stomach cancer tissues, liver cancer tissues and prostatic cancer tissues) than in normal tissues (see FIGS. 8-10).

A non-malignant cancer cell line exhibiting comparatively low ZFP91 expression level among human breast cancer cell line and stomach cancer cell line was transformed with ZFP91. ZFP91 was over-expressed in the transformed, stabilized cell line. Particularly, to investigate the functions of the gene, ZFP91 was cloned into 5′-BamHI and 3′-Xho I sites of the FLAG-tagged gene expression plasmid vector pCMV-Tag2B (Stratagene), resulting in the construction of the plasmid vector Taq2B-FLAG-ZFP91 for the expression of ZFP91 whose N-terminal was tagged with FLAG. MCF-7 and AGS cells were transformed with this vector. Western blotting was performed to measure the expression of ZFP91 by using anti-FLAG monoclonal antibody (Sigma). As a result, approximately 91 kDA sized ZFP91 protein was confirmed (FIG. 11).

The stomach cancer cell line AGS was proliferated soft agar anchorage independently to form a colony by ZFP91 (FIG. 12). Cell migration and invasion were investigated in MCF-7 and AGS cells transfected with ZFP91 expression plasmid, which were then compared with those in cells transfected with control vector. Invasion capacity comparison was performed by using Boyden Chamber (Corning Costar, Cambridge, Mass.) (FIG. 13).

It was confirmed that ZFP91 increased the expressions of proteins inhibiting apoptosis but reduced the expressions of proteins regulating cell cycle (FIG. 14).

To investigate ZFP91 expression more precisely, 330 bp DNA fragment encoding the amino acid sequence 91-200 of ZFP91 was cloned into BamHI and Ecor R1 sites of pET21β plasmid vector. By using the protein expressed in E. coli and purified therefrom as an antigen, poly clonal anti-ZFP91 antibody (constructed by the present inventors) was prepared in mice. ZFP91 expression in breast cancer cell lines and stomach cancer cell lines was confirmed by using the antibody (FIG. 15).

The functions of ZFP91 in AGS and MCF-7 cells where FLAG-ZFP91 was expressed were investigated by constructing siRNA. siRNA corresponding to the nucleotide sequence 710-730 (NCBI, NM-053023) of ZFP91 was named as ZFP91 siRNA1 (SEQ. ID. NO: 1 and NO: 2) and siRNA corresponding to the nucleotide sequence 2261-2279 of 3′ end untranslated region of ZFP91 was named as ZFP91 siRNA2 (SEQ. ID. NO: 3 and NO: 4, Quiagen).

ZFP91 siRNA oligomer was introduced into the human breast cancer cell line by using RNAiFect transfection reagent (Quiagen, Valencia, Calif.) or LIPOFECTAMIN PLUS reagent (Invitrogen, Gaithersburg, Md.). After suppressing ZFP91 expression, MTT assay was performed. As a result, when ZFP91 expression was suppressed in cells, apoptosis was induced. In fact, ZFP91 expression itself was reduced. ZFP91 siRNA was introduced into the human stomach cancer cell line AGS. As a result, most of the cells were dead and floated. From the observation on the adhered cells under microscope, typical morphology of apoptosis was observed. Therefore, it was confirmed that ZFP91 siRNA inhibited the functions of ZFP91, so that it induced apoptosis (FIG. 16). Besides, siRNA inhibited other functions of ZFP91, that is, siRNA inhibited ZFP91 not to induce p100 digestion (FIG. 21-D) and not to induce HIF-1α expression (FIG. 31 and FIG. 32-C) and suppressed the expression of HIF-1α target gene (FIG. 33).

To investigate the effect of ZFP91 on tumorigenesis and growth, the present inventors hypodermically injected the human stomach cancer cell line MKN-45 with ZFP91 over-expressed or not expressed to nude mice. As a result, in mice injected with the cell line without ZFP91 over-expression, tumorigenesis was very slow in 5 out of 6 mice. On the other hand, in mice injected with the cell line with ZFP91 over-expression, tumorigenesis was observed in every 6 mice and the size of tumor was much bigger and development speed was also very fast and angiogenesis was significantly increased as well. Blood VEGF level was significantly high in mice injected with the cell line with ZFP91 over-expression (see FIG. 17).

Since ZFP91 is the gene regulated by NF-κB, the present inventors investigated whether or not kamebakaurin (Hwang et al., Planta Medica 67, 406-410, 2001, Lee et al., J Biol Chem 277, 18411-18420, 2002; Planta Medica 70, 526-530, 2004; U.S. Pat. No. 6,894,073 B2, May 17, 2005) known as a NF-κB inhibitor having anti-inflammatory activity, celastrol (Lee et al., Biochem Pharmacol 72, 1311-1321, 2006, Korean Patent No. 040062, Sep. 14, 2004) and parthenolide (Hehner et al., J Biol Chem 273, 1288-1297, 1998; Eardie et al., Investigational New Drugs 22, 299-305, 2004) known as NF-κB inhibitors having anticancer activity and anti-inflammatory activity could inhibit ZFP91 expression. The stomach cancer cell line AGS over-expressing ZFP91 stably (FLAG-ZFP91+) was loaded in a 96-well plate at the concentration of 2×10⁵ cells/ml, followed by pre-treatment with KA (1, 10 ug/ml), celastrol (0.2, 2 ug/ml) and parthenolide (0.5, 5 ug/ml) for 30 minutes. TNF-α (50 ng/ml) was treated thereto for 3 hours. Then, cell lysate was obtained, followed by Western blot analysis using a mouse anti-FLAG antibody. The effects of these compounds on HIF-1α stabilization were also investigated in the presence of 1% oxygen. As a result, ZFP91 expression was suppressed by kamebakaurin, celastrol and parthenolide dose-dependently and HIF-1α accumulation was also significantly reduced (FIG. 37).

Cytotoxicity of kamebakaurin to cancer cells was reported (Fujita et al., Experimentia 32, 203-206, 1976), but no reports on anticancer activity in animal tests have been made so far. Thus, the present inventors transplanted the breast cancer cell line MDA-MB-435 to the mammary gland adipose tissues of nude mice and examined whether or not kamebakaurin could inhibit tumor growth and metastasis to the lung. As a result, tumor growth and metastasis to the lung were significantly inhibited by kamebakaurin at the concentration of 15 mg/kg (FIG. 38).

As explained hereinbefore, kamebakaurin capable of inhibiting ZFP91 expression and HIF-1α expression and stability suppressed tumor cell proliferation and metastasis in the mouse cancer model. This result indicates that the inhibition of ZFP91 functions leads to the suppression of tumor cell proliferation and metastasis, so that ZFP91 can be effectively used as a target molecule for the treatment of cancer.

Therefore, cancer can be suppressed by administering the effective dose of a ZFP91 inhibitor to a subject with cancer.

The effective dose can be determined by those in the art considering age, gender and weight of a subject and severity of cancer.

2. The present invention provides a method for regulating the activity of NF-κB alternative pathway and a regulator thereof.

The transcription factor NF-κB (Nuclear factor kappa B) has been known as an important molecule not only mediating immune inflammatory response but also bridging between chronic inflammation and cancer, so that it is a critical factor affecting cancer malignancy (Karin et al., Nat Rev Cancer 2,301-310, 2002; M Karin & FR Greten, Nat Rev Immunology 5, 749-759, 2005). Based on the earlier confirmation that ZFP91 is a target gene of NF-κB and its expression can be increased at mRNA level and at protein level by various NF-κB activity regulators, the present inventors further studied the mechanism of NF-κB activation.

To investigate whether ZFP91 accelerated NF-κB activation, HEK293 cells transformed with NF-κB luciferase reporter plasmid were transfected with control plasmid or ZFP91 expression plasmid, followed by investigation of the NF-κB activation. As a result, ZFP91 increased NF-κB activation dose-dependently and this effect was enhanced by TNF-α (FIG. 17). The same cells were transfected with reporter plasmid and GAL-4-DBD-p65 aa268-552 (Lee et al. Biochem Pharmacol 66, 1925-1933, 2003), the plasmid constructed to measure the transcription activity of a transcription factor. As a result, the transcription activity of p65 (RelA) was increased by ZFP91 expression plasmid dose-dependently. In the cells transfected with GAL-4-DBD-p65^{aa521-552}, the variant of Gal-4-DBD-p65, the transcription activity of p65 (RelA) was significantly increased by ZFP91 expressing plasmid dose-dependently. These results indicate that ZFP91 increases phosphorylation of Ser536 of p65 (RelA) protein which is responsible for the transcription activity of NF-κB, dose-dependently (FIG. 19).

The effect of ZFP91 on NF-κB activation induced by various NF-κB pathway activators was investigated. HEK293 cells were transfected with ZFP91 expression plasmid and TRAF2, NIK, IKKα and IKKβ expression plasmids and control plasmid. NF-κB activations therein were measured and compared. As a result, the NF-κB activation inducing effect of ZFP91 was most significant in the presence of NIK. Particularly, when ZFP91 was over-expressed together with NIK dominant negative form, NF-κB activation was not increased by ZFP91, suggesting that NIK plays an important role in ZFP91 mediated NF-κB alternative pathway activation (FIG. 20).

To examine the role of ZFP91 in NIK mediated NF-κB activation pathway, NIK alone or NIK and ZFP91 together were over-expressed in cancer cells. Then, Western blotting was performed to investigate phosphorylation of down stream molecules of NIK such as IKKα, IKKβ and p65, and degradation of IκBA, digestion of p100 and generation of p52. HEK293 cells were transfected respectively or simultaneously with FLAG-ZFP91 and c-myc-NIK expression plasmids, followed by culture. The effect of ZFP91 on NIK mediated phosphorylation of p65 (RelA) Ser536, degradation of IκBA, phosphorylation of IKKα, IKKβ and phospho-p38 MAPK was investigated by Western blotting. As a result, ZFP91 was confirmed to increase phosphorylation of NIK downstream molecules IKKα, p38 and p65, and increase p52 by digesting p100. However, ZFP91 did not increase phosphorylation of IKKβ and was not involved in degradation of IκBA, suggesting that ZFP91 is more like an important molecule in NIK mediated alternative pathway (FIG. 21).

The activation of NF-κB begins with recruiting of various adaptor proteins such as TRAF and RIP (receptor interacting protein) to the receptor and it is suggested that alternative pathway is activated by the interaction of NIK with TRAF2 (Grech et al., Immunity 21, 629-642, 2004). Based on that, interaction between ZFP91 and TRAF2, which is known to be involved in NIK activation was investigated. Particularly, the extracts of HEK293T cells transfected with myc-NIK and FLAG-ZFP91 or HA-TRAF2 and FLAG-ZFP91 were immuno-precipitated using anti-FLAG, anti-NIK or anti-HA antibody, followed by Western blotting using anti-FLAG, anti-HA or anti-NIK antibodies. To confirm whether or not these three molecules formed a complex, ZFP91, NIK and TRAF2 were all over-expressed in HEK293 cells, followed by immuno-precipitation with ZFP91 to investigate the conjugation of NIK to TRAF2. As a result, all three molecules formed a complex together (FIG. 22).

Then, which domain of ZFP91 was important in the interaction with NIK was investigated. NIK and ZFP91 deletion mutants were co-expressed in HEK293 cells, followed by immuno-precipitation. As a result, interaction did not occur without Zinc finger domain (see FIG. 22). This result indicates that Zinc finger domain plays an important role in the interaction between ZFP91 and NIK. Next, which domain of NIK was important in the interaction with ZFP91 was also investigated. ZFP91 and NIK deletion mutants were co-expressed in HEK293 cells, followed by immuno-precipitation. Western blotting was performed using each corresponding antibody. As a result, it was confirmed that kinase domain of NIK was important for the interaction with ZFP91 (FIGS. 22, 23 and 24).

According to the previous reports, for the stabilization and activation of NIK, TRAF3 is involved in NIK degradation by ubiquitination (Liao et al., J Biol Chem 279, 26243-26250, 2004) and the activity of p52/RelB mediated by NF-kB2 (p100) degradation is high in TRAF2 (Marin et al., Nature 385, 540-544, 1997) deficient B cells (Grech et al., Immunity 21, 629-642, 2004). However, there has been no report on the effect of TRAF2 on NIK. So, the present inventors investigated whether or not ZFP91 induced poly-ubiquitination of NIK protein in HEK293 cells. HEK293 cells were transfected with myc-NIK only, myc-NIK together with HA-Ubiquitin, or myc-NIK together with HA-Ubiquitin and Flag-ZFP91. Those cells were cultured and the cell extracts were immuno-precipitated using anti-NIK antibody, followed by Western blotting using anti-HA-Ubiquitin antibody. Some of cell lysate was taken to measure expression of input proteins before the immuno-precipitation. As a result, poly-ubiquitination of myc-NIK by ZFP91 was confirmed (FIG. 25-A).

To examine how NIK ubiquitination by ZFP91 was regulated in ZFP91 deletion mutant, myc-NIK was introduced into HEK293 cells with full leng of ZFP91, HA-Ubiquitin, HA-ubiquitin and FLAG ZFP91, or HA-Ubiquitin and FLAG ZFP91 mutant deletd zinc finger domains. Those cells were cultured and the cell extracts were immuno-precipitated using anti-NIK antibody. Western blotting was performed by the same manner as described above. As a result, poly-ubiquitination was observed only in the cell group introduced with full length of ZFP91 with ubiquitin (FIG. 25-B). In vitro ubiquitination was also examined with FLAGZFP91 and MYCNIK purified from the HEK293 cells using commercially available ubiquitin activating enzyme E1, ubiquitin transferase E2, and ubiquitin. As a result, ubiquitination was significant in the presence of NIK (FIGS. 26-29).

In this experiment, ubiquitinated NIK was accumulated in the absence of MG-132, the proteasome inhibitor, suggesting that ZFP91 induced NIK ubiquitination was involved in the activation of NF-κB alternative pathway.

The above results indicate that ZFP91 expression is regulated by NF-κB and up-regulated ZFP91 stimulates the transcription activity of NF-κB, in particular the activation of the alternative pathway, in return. So, it was presumed that ZFP91 induces cancer malignancy by regulating the expression of a target gene involved in cell proliferation of NF-κB alternative pathway. It is proved that NF-κB alternative pathway is a critical factor in inflammation and immune responses, precisely in the differentiation and growth and functions of immune cells.

The activation of NF-κB alternative pathway can be regulated by controlling the activation or expression of ZFP91 and therefore molecules capable of regulating the activation or expression of ZFP91 can be effectively used for the treatment of cancer, inflammation and immune disease.

3. The present invention provides a method for regulating the expression and functions of HIF-1α and regulating the expression of major HIF-1 target proteins such as VEGF and EPO and a regulator thereof.

NF-κB stabilizes HIF-1α which is a transcription factor playing an important role in cancer malignancy and metastasis and increases the expression of angiogenesis factor such as VEGF (Jung et al., FASEB J. express article 10.1096/fj.03-0329fje. published online Sep. 4, 2003; Zhou et al., Cancer Res 64, 9041-9048, 2004). However, the mechanism of the above has not been disclosed, yet. In the earlier experiment using pGL3-ZFP91prom-LUC constructed by using two NF-κB binding transcription regulation sites of ZFP91, molecules inducing hypoxia inducible factor-1 such as DFO (deferoxamine) and CoCl₂ increased the expression of a reporter gene (FIG. 5-E). Herein, the effect of ZFP91 on HIF-1α stabilization and activation was investigated.

First, HIF-1 dependent reporter assay plasmid was introduced into AGS, HT-29 and Hep3B cells, and then these cells were transformed with ZFP91 expression plasmid or control plasmid, followed by overnight culture. As results HIF-1 activation and stabilization in every cells by ZFP91 dose-dependently regardless of partial oxygen pressure, were confirmed by hypoxia response element (HRE) dependent reporter assay (FIGS. 30-A and B) and Western blot analysis using anti-HIF-1α antibody (FIG. 30-C). The increase of vascular endothelial growth factor (VEGF) expression was also confirmed by Northern blotting (FIG. 30-D). The above effect of ZFP91 was inhibited by ZFP91 siRNA, though (FIGS. 31 and 32). ZFP91 increased the expressions of HIF-1 target genes regardless of partial oxygen pressure. This effect of ZFP91 was inhibited by siRNA, confirmed by RT-PCR examining and comparing the expressions of HIF-1α target gene mRNA in between AGS cells and AGS cells transfected with ZFP91 (FIG. 33).

Next, it was investigated whether ZFP91 was capable of stabilizing HIF-1α playing an important role in cancer malignancy and metastasis.

HEK293 cells transfected with each plasmid for HA-VHL, FLAG-ZFP91 and GAL4-HIF-1α were cultured for 48 hours. The cell extracts were immuno-precipitated using anti-FLAG antibody, anti-HA antibody, or anti-GAL-4 antibody, followed by Western blotting using anti-HA antibody, anti-FLAG antibody and anti-HIF-1αantibody. As a result, it was confirmed that ZFP91 formed a complex with VHL and HIF-1α (FIG. 34).

It was investigated whether ZFP91 was involved in HIF-1α ubiquitination as it was in NIK ubiquitination. HA-Ubiquitin, GAL4-HIF-1α, and VHL were combined and expressed in HEK293 cells in the presence of the proteasome inhibitor MG132, followed by immuno-precipitation using anti-GAL4 antibody. Then, Western blotting was performed using anti-HA antibody. As a result, ZFP91 was confirmed to increase HIF-1α ubiquitination dose-dependently (FIG. 35).

ZFP91 can reduce the expression of a tumor suppressor gene pVHL (Von Hippel-Lindau), which degrade HIF-1α and its homologue HIF-2α by ubiquitin ligase activity with Elongin B and C, Rbx 1 and Cullin 2 but increase the expression of UCP (Jung et al., Nature Med 12, 809-816, 2006) reported as a protein degrading pVHL via ubiquitination (FIG. 36).

The above results indicate that HIF-1α related various pathological phenomena can be improved by regulating the expression or stabilization of HIF-1α by controlling the expression or functions of ZFP91. As explained hereinbefore, the present inventors confirmed that ZFP91 siRNA suppressed the expression and stabilization of HIF-1α and NF-κB inhibitors also suppressed the expression of ZFP91 and the expression of HIF-1α induced under hypoxic condition. Therefore, the suppression of the expression or functions of ZFP91 might bring anticancer effect and inhibition of metastasis.

ZFP91 increased the expression of VEGF or EPO by HIF-1α activation regardless of partial oxygen pressure and this activity was demonstrated at mRNA and protein levels in xenograft models, suggesting that the molecule increasing ZFP91 expression can be effectively used for the treatment of ischemic diseases.

4. The present invention provides a ZFP91 inhibitor.

ZFP91 activation can be inhibited by the materials suppressing ZFP91 transcription, ZFP91 mRNA translation or ZFP91 protein function.

The material suppressing transcription can be a promoter known to regulate ZFP91 transcription, an enhancer, a protein or a compound binding to a transcription regulator to be bound to the promoter.

The material suppressing mRNA translation can be a low molecular compound or siRNA prepared by using antisense nucleotide sequence or RNAi technique.

The material suppressing ZFP91 protein function can be a peptide, an antibody, a compound and peptide mimetics binding to the protein.

Particularly,

1) RNAi (siRNA)

RNA interference (RNAi) is a post transcriptional gene silencing mechanism in which double stranded RNA (dsRNA) corresponding to ZFP91 gene was introduced into cells or organisms. By the RNAi effect, multiple cell divisions occur constantly before gene expression recovery. Therefore, RNAi is the most powerful method to produce knock-out or knock-down models. RNAi was confirmed to be very effective in human cells including embroyonic kidney and HeLa cells (Elbashir et al., Nature, 411,494-498, 2001).

Gene silencing related RNAi technique is based on standard molecular biological methods. Double stranded RNA corresponding to a target gene supposed to be inactivated can be prepared by the standard method, for example, this can be generated by simultaneous transcription with two strands of a template DNA using T7 RNA polymerase. Preparation kit of ds RNA used for RNAi can be any commercial product (ex. product of New England Biolabs, Inc.). The method for transfecting dsRNA or plasmid designed to produce dsRNA can be any conventional method well known to those in the art.

Suppression of ZFP91 expression by using antisense nucleotide sequence of ZFP91 was reported previously (Unoki et al., Intern J Oncol 22, 1217-1223, 2003) but inhibition of ZFP91 expression by using siRNA has not been reported. Therefore, 710-730 nucleotides region (caggtg gcattagtag tgaa) of ZFP91 sequence (NCBI, NM_{—}053023) was named as ZFP91 siRNA 1, 2261-2279 nucleotides region (gcggcacact tatcttcaa) of 3′ non-translational region of ZFP91 was named as ZFP91 siRNA 2, and siRNAs of these regions were prepared (QIAGEN). Suppression of ZFP91 expression by these siRNAs was confirmed in various cancer cells (FIG. 31-C and FIG. 32-C).

2) Peptide Mimetics

Mimetics (ex. peptide or peptide drug) designed to suppress the protein binding domain of ZFP91 polypeptide was confirmed to inhibit ZFP91 binding to HIF, VHL, NIK, and TRAF2.

Major residues of non-hydrolyzable peptide analog can be produced by using β-turn dipeptide core (Nagai et al. Tetrahedron Lett 26:647, 1985), keto-methylene sheudopeptides (Ewenson et al. J Med Chem 29:295, 1986; and Ewenson et al. in Peptides: Structure and Function (Proceedings of the 9th American Peptide Symposium) Pierce Chemical Co. Rockland, Ill., 1985), azepine (Huffman et al. in Peptides: Chemistry and Biology, G. R. Marshall ed., ESCOM Publisher: Leiden, Netherlands, 1988), benzodiazepine (Freidinger et al. in Peptides; Chemistry and Biology, G. R. Marshall ed., ESCOM Publisher: Leiden, Netherlands, 1988), β-aminoalcohol (Gordon et al. Biochem Biophys Res Commun 126:419 1985) and substituted gamma lactam ring (Garvey et al. in Peptides: Chemistry and Biology, G. R. Marshell ed., ESCOM Publisher: Leiden, Netherlands, 1988).

Peptide mimetics inhibiting protein functions of ZFP91 can be synthesized with domains important for binding with ZFP91, NIK, TRAF2, HIF and VHL and the modified region after ZFP91 transcription.

5. The present invention provides a method for increasing the activity of NF-κB alternative pathway by increasing the activity of ZFP91, suppressing the expression of cell cycle inhibiting protein, increasing the stability of HIF-1α and promoting the expressions of VEGF and EPO.

As explained hereinbefore, when ZFP91 is over-expressed, NIK mediated NF-κB alternative pathway is activated and HIF-1α is stabilized (see FIGS. 30, 31 and 32), so that angiogenesis factors such as VEGF (vascular endothelial growth factor) and EPO (erythropoietin) are up-regulated by HIF-1α (FIG. 33-A). Therefore, the increase of the activity of ZFP91 results in the increase of the expression of VEGF, which is mediated by an agent inducing ZFP91 mRNA expression by interacting with a ZFP91 promoter or a plasmid or a virus gene carrier inducing ZFP91 expression.

The ZFP91 activity enhancer includes an agent inducing ZFP91 mRNA expression by interacting with a ZFP91 promoter (Korean Patent No. 2003-0013795), a plasmid inducing ZFP91 expression (Korean Patent No. 10-0375890) or a virus gene carrier (Korean Patent No. 2001-0006460).

When ZFP91 gene is over-expressed, VEGF and EPO expressions are promoted. So, such a method that brings similar effect to ZFP91 over-expression, that is direct insertion of ZFP91 protein or insertion of ZFP91 expression plasmid to a subject, can promote VEGF and EPO expressions.

6. The present invention provides an angiogenesis enhancer containing a ZFP91 activity enhancer, an expression vector containing ZFP91 gene or a ZFP91 protein as an active ingredient. Over-expression of ZFP91 of the present invention results in the increase of HIF-1α level (see FIGS. 30, 31 and 32) and VEGF and EPO expressions (see FIGS. 17, 30-D and 33) to stimulate angiogenesis.

It is well known that up-regulation of VEGF is effective in the treatment of ischemic vascular disease (Yla-Herttuala S and Alitalo K. Nat. Med., 9, 694-701, 2003; Khan T A et al., Gene Ther. 10, 285-91, 2003). So, the angiogenesis promoter containing the expression vector for ZFP91 gene to increase VEGF expression can be effectively used for the treatment of vascular diseases such as critical limb ischemia (CLI) that needs to get limb amputation caused by poor blood vessels, and coronary artery disease (CAD) which is not suitable for surgical treatment. In addition, ZFP91 can also be effectively used for gene therapy for incurable disease including dementia, amyotrophic lateral sclerosis (ALS), diabetic neuropathy and stroke caused by poor blood supply.

7. The present invention provides a method for screening a ZFP91 activity regulator.

1) Using ZFP91-Promoter-Reporter Vector

The ZFP91 promoter-reporter vector is useful for the method of screening anticancer candidates comprising the step of selecting test compounds. The present invention provides a method for screening a ZFP91 activity regulator (gene, protein, low molecule compound, natural substance, natural extract, siRNA, miRNA and nucleic acid, etc) to measure the expression of ZFP91 gene by using transformed cells prepared by transfecting mammalian cells with the ZFP91-promoter-reporter vector. The interactions of RNA-RNA, DNA-DNA, DNA-RNA, RNA-protein, RNA-compound, DNA-protein, and DNA-compound are largely confirmed by hybridization experiment measuring the in vitro binding between the said gene and the activity regulator candidates; Northern blotting performed after reacting mammalian cells with inhibitor candidates; semi-quantitative or quantitative PCR and real time PCR to measure ZFP91 gene expression; and a method in which a reporter gene is conjugated to ZFP91 gene, which is introduced into cells and reacted therein with inhibitor candidates to measure the reporter gene expression. In this screening, the ZFP91 expression or activity regulator candidate can be selected by the conventional method. Particularly, a material presumed to have capability of down- or up-regulating ZFP91 for example, an individual nucleic acid, a protein, other extracts of natural substances, can be selected as the candidate.

The candidates having inhibitory or increasing activity of gene expression or protein stability selected by the screening method of the present invention can be anticancer agent candidates or angiogenesis inducers. These candidates will be acting as a leading material in the development of an anticancer agent. Particularly, these leading materials can be modified in their structures or optimized to be effective in the inhibition or enhancement of proteins expressed by ZFP91, leading to the development of a novel anticancer agent.

For example, the down stream of −1105 and −1664 regions of 5′-upstream of ZFP91, which contain two NF-κB consensus sequences, are linked with a reporter gene, luciferase, to construct NF-kB dependent reporter plasmid based on pGL3Basic vector. Host cells are transfected with the vector and then treated with NF-kB activators such as TNF-α, LPS and PMN, followed by measuring the activation of the reporter gene, luciferase to screen a ZFP91 expression regulator (see FIG. 3-C and FIG. 5).

2) Cell-based screening method is provided, which comprises the step of selecting test compounds using ZFP91 transformants prepared from ZFP91 low expression mother cells and an expression vector for mammals or adenovirus and lentivirus to select anticancer agent candidates. The screening method is based on the comparison of cytotoxicity, cell motility, invasiveness, colony formation on agar, NF-κB activity and HIF activity between mother cells and the transformants (see FIGS. 11, 12, 13 and 16).

3) Method using fluorescent protein fused ZFP91

ZFP91 or specific regions of ZFP91 are fused with fluorescent proteins such as GFP, YFP and RFP, resulting in the construction of an expression vector. Mammalian cells transfected with the expression vector can be used for cell-based screening to screen ZFP91 activity regulator candidates containing the step of selecting test compounds. The test compound can be directly treated to the cells transformed with the fluorescent protein fused ZFP91 or the test compound can be mixed with a stimulator inducing ZFP91 expression and the mixture is treated to the cells. Fluorescence changes are measured by various methods to select ZFP91 activity regulator candidates.

4) Method for inhibiting the interaction between ZFP91 and NIK, TRAF, IKKα and HIF-1α

A screening method using protein-protein interaction, particularly between ZFP91 and NIK, TRAF, IKKα and HIF-1α, is provided to screen a ZFP91 activity regulator. In vivo and ex vivo reaction between ZFP91 protein and its activity regulator is induced by using full length ZFP91 or a specific region for protein binding, for example Zinc Finger Domain of ZFP91 (FIG. 23) or a specific region of a protein binding to full length NIK, TRAF, IKKα, HIF-1α or a specific region binding to ZFP91 such as NIK kinase domain (FIG. 24). Then, a compound regulating the interaction between ZFP91 and the above proteins can be screened by the method measuring the interaction between proteins and yeast two-hybrid method.

FRET (fluorescence resonance energy transfer) between the full length of or a specific region of ZFP91 and the full length or a specific region of the binding proteins enables the screening method for a ZFP91 activity regulator. For example, the full length of ZFP91 or its zinc finger domain binding NIK is used for the construction of an expression vector to produce YFP fusion protein. The full length of NIK or a specific region interacting with ZFP91 is used for the construction of an expression vector to produce CFP fusion protein. Mammalian cells are transfected with the vectors and the fusion proteins are expressed therein. Energy transfer by the interaction between ZFP91 and NIK is confirmed. Irradiation is executed at 433 nm and fluorescence is detected not at 476 but at 527 nm. At this time, if there were a compound inhibiting the binding, fluorescence at 527 nm would be reduced. So, cell-based screening method for screening ZFP91 activation regulator candidates is provided by using FRET between ZFP91 and binding proteins such as NIK, IKKα and TRAF. A test compound can be directly added to the cells transformed with ZFP91 fused with a specific fluorescent protein or a binding protein binding to a specific region of ZFP91 fused with a specific fluorescent protein or a specific region of the binding protein. Fluorescence changes can be investigated by various methods to screen ZFP91 activity regulator candidates.

To screen a candidate that inhibits the binding of ZFP91 with NIK protein, a test compound is treated to the cells expressing both ZFP91 and NIK simultaneously and then the first fluorescent material labeled anti-ZFP91 antibody and the second fluorescent material labeled anti-NIK antibody are treated thereto, followed by measuring the fluorescences of the first fluorescent material and the second fluorescent material. At this time, if the fluorescent intensity of the first fluorescent material is overlapped that of the second fluorescent material, it means ZFP91 protein is conjugated to NIK protein. So, by investigating the overlapping of fluorescent intensity, whether or not the test compound inhibits the binding of ZFP91 protein to NIK protein can be decided.

8. The present invention provides a method for diagnosing cancer and confirming the treatment result or prognosis comprising the step of measuring the expression of ZFP91 by using one or more materials reacting with ZFP91 in diagnostic samples obtained from a subject and a diagnostic kit using the same.

1) The present invention provides a method for diagnosing cancer containing the step of measuring the expression of ZFP91 in a diagnostic sample of a subject.

The increased ZFP91 expression, higher than normal, in a diagnostic sample indicates that the subject might have cancer. In this diagnostic method, measuring the expression of ZFP91 is performed by the same manner as described in the above screening method for determining the expression or activation of ZFP91 gene.

2) The present invention provides a method for evaluating cancer treatment effect of the method containing the step of measuring the expression of ZFP91 in a diagnostic sample of a subject who is either treated or is under the cancer treatment.

Normal ZFP91 expression level in a diagnostic sample indicates that the cancer treatment is successful, while the abnormally increased ZFP91 level indicates that the cancer treatment has to be going on.

3) The present invention provides a method for evaluating prognosis of cancer containing the step of measuring the expression of ZFP91 in a diagnostic sample of a subject with cancer.

In this step, normal ZFP91 level indicates the prognosis will be good, while abnormally increased ZFP91 level indicates the prognosis is not good.

4) The diagnostic kit for cancer of the present invention can additionally include one or more materials reacting ZFP91 and a reagent for detecting reactants and protocol thereof. For example, one or more materials reacting ZFP91 can be RNA or DNA complement to ZFP91 RNA or RNA or antibody binding to ZFP91 protein. The reagent for detecting reactants can be nucleic acid or protein labeling and coloring reagents. For example in this invention, 330 bp of the DNA fragment encoding 91-200 amino acids of ZFP91 of the present invention was cloned into BamHI and EcoRI sites of pET21a plasmid vector, which was expressed in E. coli BL21. The expressed protein was purified and used as an antigen for the production of polyclonal anti-ZFP91 antibody in mice. This antibody was used for the expression of ZFP91 in various cancer cell lines including breast cancer and stomach cancer cell lines. This antibody can be used for measuring ZFP91 level in serum of a subject.

### ADVANTAGEOUS EFFECT

ZFP91 has oncogene like activity, which is increasing the expressions of apoptosis inhibiting proteins by activating NIK and IKKα, the important proteins involved in the alternative pathway of NF-κB activation, and increasing the expressions of proteins regulating cell cycle. Therefore, the anticancer agent of the present invention containing a ZFP91 inhibitor can be effectively used for the treatment of cancer by suppressing cancer malignancy mediated by NF-κB activation pathway among cancer malignancy related signal transduction pathways. The ZFP91 inhibitor can also be used for the treatment of angiogenesis related diseases including diabetic retinopathy and arthritis caused by the up-regulation of VEGF induced by HIF-1 under hypoxic condition. As explained hereinbefore, when ZFP91 expression is increased, the tumor suppressor protein VHL is degraded and thereby HIF-1α is stabilized. Then, the expressions of proteins such as angiogenesis promoters including VEGF mediated by HIF-1 are also promoted. So, inhibition of ZFP91 activation in cancer cells results in the promotion of degradation of HIF-1α and inhibition of angiogenesis promoter such as VEGF, so that tumor growth and metastasis is accordingly inhibited. Therefore, the ZFP91 inhibitor of the present invention can be used as an anticancer agent. In the meantime, over-expression of ZFP91 results in HIF-1α stabilization, and thereby angiogenesis promoter such as VEGF is up-regulated. Therefore, the ZFP91 up-regulator can be effectively used for gene therapy for treating those having ischemic diseases caused by poor blood vessels, for example patients with incurable diseases including critical limb ischemia (CLI) requiring limb amputation because there is no other way to be treated, coronary artery disease (CAD) that cannot be treated with surgery, dementia caused by poor blood supply, amyotrophic lateral sclerosis (ALS), diabetic neuropathy, stroke, etc.

### DESCRIPTION OF DRAWINGS

The application of the preferred embodiments of the present invention is best understood with reference to the accompanying drawings, wherein:

FIG. 1 is a diagram illustrating the expressions of candidate genes regulated by kamebakaurin, confirmed by RT-PCR.

FIG. 2 is a diagram illustrating the expressions of candidate genes regulated by kamebakaurin, confirmed by Northern blotting.

FIG. 3 is a diagram illustrating the amino acid sequence of ZFP91 (A), nucleotide sequence of NF-κB binding site on promoter (B), and ZFP91 promoter deletion mutant (C).

FIG. 4 is a diagram illustrating the identification of κB region of ZFP91 promoter by electrophoretic mobility shift assay (EMSA). KB1 is identified by the oligonucleotide synthesized using the sequence from −1675 to −1664 of FIG. 3B and KB2 is identified by the oligonucleotide synthesized using the sequence from −1114 to −1105 of FIG. 3B.

FIG. 5 is a diagram illustrating the investigation of the effect of various NF-κB activators on the transcription activity of ZFP91 promoter by reporter assay using ZFP91 promoter deletion mutants (FIG. 3-C). (A) regulation of transcription activity of ZFP91 by the NF-κB activators TNF-α, IL-1β and PMA, (B) regulation of transcription activity of ZFP91 by RelA (p65) over-expression, (C) regulation of transcription activity of ZFP91 by IKKα over-expression, (D) regulation of transcription activity of ZFP91 by IKKβ over-expression, and (E) regulation of transcription activity of ZFP91 by DFO (deferoxamine), CoCl₂ and H₂O₂.

FIG. 6 is a diagram illustrating the effect of various NF-κB activators on ZFP91 expression. (A) ZFP91 expression pattern in MCF-7 cells stimulated by TNF-α, PMA, IL-1β or H₂O₂, and (B) ZFP91 expression pattern in MCF-7 cells transfected with control plasmid or NIK, IKKα, IKKβ and p65 expression plasmids.

FIG. 7 is a diagram illustrating the ZFP91 mRNA expressions in breast cancer cell lines (A) and stomach cancer cell lines (B).

FIG. 8 is a diagram illustrating the ZFP91 mRNA expressions in normal stomach tissues and stomach cancer tissues, detected by in situ hybridization.

FIG. 9 is a diagram illustrating the ZFP91 mRNA expressions in normal liver tissues and liver cancer tissues, detected by in situ hybridization.

FIG. 10 is a diagram illustrating the ZFP91 mRNA expressions in normal prostatic tissues and prostatic cancer tissues, detected by in situ hybridization.

FIG. 11 is a diagram illustrating the ZFP91 expressions in MCF-7 cells and AGS cells transfected with FLAG-ZFP91.

FIG. 12 is a diagram illustrating the anchorage independent growth of AGS cells transfected with ZFP91, detected by colony formation in soft agar.

FIG. 13 is a diagram illustrating the effect of ZFP91 on invasion of MCF-7 and AGS cells. (A) comparison of invasion capacities among MCF-7 cells, MCF-7 cells transfected with control plasmid, and MCF-7 cells transfected with ZFP91, and (B) comparison of invasion capacities among AGS cells, AGS cells transfected with control plasmid, and AGS cells transfected with ZFP91.

FIG. 14 is a diagram illustrating the effect of ZFP91 on the expressions of apoptosis inhibiting proteins and cell cycle regulator proteins, confirmed by Western blotting. (A) Expressions of the NF-κB target genes/apoptosis inhibiting proteins cIAP1 and cIAP-1 were increased by ZFP91, and (B) Expressions of the cell cycle inhibiting proteins p27 and p21 were suppressed by ZFP91.

FIG. 15 is a diagram illustrating the effect of anti-ZFP91 antibody prepared by using ZFP91 protein as an antigen. (A) The effect of anti-ZFP91 antibody on the expressions of ZFP91 in 293 cells, HT-29 cells and U937 cells transfected with control plasmid or FLAG-ZFP91, and (B) The effect of anti-ZFP91 antibody on the expressions of ZFP91 in various breast cancer cell lines and stomach cancer cell lines.

FIG. 16 is a diagram illustrating the effect of ZFP91 on apoptosis. (A) The result of MTT assay with MCF-7 cells transfected with control siRNA oligomer or ZFP91 siRNA oligomer, wherein ZFP91 siRNA induced apoptosis, (B) The result of immunoblotting with AGS cells transfected with control siRNA and ZFP91 siRNA by using ZFP91 antibody, wherein ZFP91 expression was reduced, and (C) The changes of cell morphology in adhered and surviving cells, observed under microscope.

FIG. 17 is a diagram illustrating the effect of ZFP91 on tumorigenesis and growth in MKN-45 cells. (A) is a photograph illustrating the tumorigenesis after 4 weeks from the transplantation of MKN-45 cells transfected with control plasmid or ZFP91 into nude mice, (B) is a graph illustrating the comparison of tumor sizes, and (C) is a diagram illustrating the comparison of blood VEGF levels.

FIG. 18 is a diagram illustrating the promotion of NF-κB activation by ZFP91. (A) ZFP91 expression vector dose-dependent promotion of NF-κB activation in HEK293 cells transfected with NF-κB luciferase reporter plasmid, and (B) The effect of TNF-α in HEK293 cells transfected with control plasmid used in (A) and ZFP91 respectively.

FIG. 19 is a diagram illustrating the ZFP91 activity to increase transcription activity of p65 (RelA) protein by Ser536 phosphorylation. (A) ZFP91 expression vector dose-dependent promotion of NF-κB activation in HEK293 cells transfected with NF-κB luciferase reporter plasmid, (B) ZFP91 expression vector dose-dependent promotion of p65 (RelA) transcription activity along with the promotion of NF-κB activation when the cells of (A) were transfected with GAL-4-DBD-p65 plasmid, (C) ZFP91 expression vector dose-dependent increase of p65 (RelA) Ser 536 phosphorylation in MCF-7 cells, confirmed by Western blotting using anti-phospho(ser⁵³⁶)-p65 antibody, and (D) p65 transcription activation domain 1 (TA1) introduced into GAL4-p65 mutant.

FIG. 20 is a diagram illustrating the effect of ZFP91 on NF-κB activity induced by various signal molecules of NF-κB pathway. (A) Comparison of NF-κB activations in HEK293 cells transfected with ZFP91 expression plasmid, TRAF2, NIK, IKKα and IKKβ expression plasmids, respectively, and control plasmid, and (B) Comparison of NIK effect shown in (A) with NIK dominant negative form (DN).

FIG. 21 is a diagram illustrating that ZFP91 is an important factor in NIK mediated NF-κB activation. (A) HEK293 cells were transfected with FLAG-ZFP91 and c-myc-NIK expression plasmid respectively or simultaneously and cultured. The effect of ZFP91 on NIK mediated p65 (RelA) Ser536 phosphorylation, IkBα degradation, IKKα, IKKβ, and phospho-p38 MAPK phosphorylation, was detected by Western blotting using each corresponding antibody, (B) Effect of ZFP91 on the processing of NF-κB2 molecule to p52 which is an important factor of NF-κB alternative pathway, (C) p52 production increased ZFP91-dose-dependently in HEK293 cells transfected with c-myc-NIK, and (D) p52 and its precursor p100 reduced in MDA-MB-231 cells by the treatment of ZFP91 siRNA.

FIG. 22 is a diagram showing the result of Western blotting illustrating that ZFP91 forms a complex with TRAF2 and NIK and interacts with them. (A) Extracts of HEK293 cells transfected with myc-NIK and FLAG-ZFP91 or HA-TRAF2 and FLAG-ZFP91 were immuno-precipitated with anti-FLAG, anti-NIK or anti-HA antibody, followed by Western blotting using anti-FLAG antibody, and (B) Extracts of HEK293 cells transfected with HA-TRAF2, myc-NIK and FLAG-ZFP91 were immuno-precipitated with anti-FLAG antibody, followed by Western blotting using anti-HA, anti-NIK, and anti-FLAG antibodies.

FIG. 23 is a diagram showing the result of Western blotting illustrating that NIK forms a complex with Zinc Finger Domain of ZFP91. (A) Extracts of HEK293 cells transfected with myc-NIK and FLAG-ZFP91 mutant were immuno-precipitated with anti-FLAG antibody, followed by Western blotting using anti-NIK antibody, and (B) Human ZFP91 deletion mutants.

FIG. 24 is a diagram showing the result of Western blotting illustrating that ZFP91 forms a reciprocal complex with the fragment containing kinase domain of NIK. (A) Extracts of the cells transfected with FLAG91 and NIK mutant plasmid were immuno-precipitated with anti-FLAG antibody, followed by Western blotting using each antibody, and (B) NIK deletion mutants.

FIG. 25 is a diagram showing the result of Western blotting illustrating that ZFP91 induces E oly-ubiquitination of NIK protein in HEK293 cells. (A) HEK293 cells were transfected with various combinations of Myc-NIK HA-Ubiquitin, FLAG-ZFP91 and control plasmid. After incubation extracts of the cells were immuno-precipitated with anti-NIK antibody, followed by Western blotting using anti-HA antibody or anti-myc antibody, and (B) Myc-NIK was introduced into HEK293 cells with full length of ZFP91, HA-ubiquitin and FLAG ZFP91, or HA-Ubiquitin and FLAG ZFP91 mutant deletd zinc finger domains. After incubation extracts of the cells were immuno-precipitated with anti-NIK antibody, followed by Western blotting using anti-HA antibody, anti-myc antibody and anti-NIK antibody.

FIG. 26 is a diagram illustrating the in vitro ubiquitination. ZFP91 has an intrinsic ubiquitin ligase activity and required Ubc13 as an E-2 enzyme. In in vitro ubiquitination assay, ZFP91 and NIK purified from HEK293 cells and ZFP91 C-terminal containing zinc finger domains purified from E. coli were mixed with a reaction mixture containing ubiquitin, E1 enzyme, ATP, E2 enzyme mixture or Ubc13/Mms2. ZFP91 ubiquitination was auto-stimulated E2 enzyme Ubc13 dependently.

FIG. 27 is a diagram illustrating the in vitro ubiquitination. ZFP91 is auto-ubiquitinated Ubc13-dependently (left) and ZFP91 ubiquitinate NIK and the ubiquitination was increased in the presence of NIK (right).

FIG. 28 is a diagram illustrating the in vitro ubiquitination. ZFP91 assembled ubiquinone chains ZFP91 C term dependently.

FIG. 29 is a diagram illustrating the in vitro ubiquitination. The ubiquinone chains assembled by ZFP91 via ZFP91 C term moved to NIK.

FIG. 30 is a diagram illustrating the effect of ZFP91 on the expression and activation of HIF-1α. The ZFP91-dependent activation of HIF-1 at normoxia and hypoxia (A) was confirmed by HRE (hypoxia response element) dependent reporter assay in AGS cells (A-1), HT-29 cells (A-2), and Hep3B cells (A-3). (B) ZFP91-dose-dependent HIF-1 activation in AGS cells, confirmed by reporter assay, (C) ZFP91-dose-dependent HIF-1α expression in AGS cells, confirmed by Western blotting, and (D) ZFP91 dependent HIF-1 target gene VEGF expression in AGS cells, confirmed by Northern blotting.

FIG. 31 is a diagram illustrating that ZFP91 siRNA inhibited HIF-1 activation and HIF-1α expression in AGS cells and AGS cells over-expressing ZFP91. (A) Suppression of HIF-1α activation by ZFP91 siRNA in AGS cells, confirmed by reporter assay, (B) Suppression of HIF-1α activation by ZFP91 siRNA in AGS cells transfected with ZFP91 expression plasmid, confirmed by reporter assay, and (C) Suppression of HIF-1α expression by ZFP91 siRNA in AGS cells and AGS cells transfected with ZFP91, confirmed by Western blotting.

FIG. 32 is a diagram illustrating the expression of HIF-1α protein in AGS cells (control cells) and AGS cells over-expressing ZFP91 (ZFP91 cells). (A) The expression of HIF-1α protein was examined over the time by Western blotting in the presence of 1% oxygen, (B) The expression of HIF-1α protein was examined by Western blotting in the presence of the proteasome inhibitor MG-132 under normoxic condition, and (C) The expression of HIF-1α protein was inhibited by ZFP91 siRNA in AGS cells, confirmed by Western blotting.

FIG. 33 is a diagram illustrating the HIF-1α target gene expression in AGS cells. (A) Comparison of HIF-1α target gene mRNA expressions in AGS cells and AGS cell expressing ZFP91 by RT-PCR, and (B) Inhibition of HIF-1α target gene mRNA expression by ZFP91 siRNA.

FIG. 34 is a diagram showing the result of Western blotting illustrating that ZFP91 forms a complex with pVHL and HIF-1α. (A) HEK293 cells transfected with HA-VHL and FLAG-ZFP91 expression plasmids were cultured for 48 hours. Extracts of the cells were immuno-precipitated with anti-FLAG and anti-HA antibody, followed by Western blotting using anti-HA antibody and anti-FLAG antibody, (B) HEK293 cells transfected with GAL4-HIF-1α and FLAG-ZFP91 expression plasmids were cultured for 48 hours. Extracts of the cells were immuno-precipitated with anti-GAL-4 antibody, followed by Western blotting using anti-FLAG antibody and anti-HIF-1α antibody, and (C) HEK293 cells transfected with HA-VHL, FLAG-ZFP91 and GAL4-HIF-1α expression plasmids were cultured for 48 hours. Extracts of the cells were immuno-precipitated with anti-FLAG antibody, followed by Western blotting using anti-HA antibody and anti-FALG antibody.

FIG. 35 is a diagram showing the result of Western blotting to examine the effect of ZFP91 on the enhancement of HIF-1α ubiquitination. HA-Ub, GAL4-HIF-1α, and pVHL were combined and expressed in HEK293 cells in the presence of the proteasome inhibitor MG132, followed by immuno-precipitation using anti-GAL4 antibody. Then, Western blotting was performed using anti-HA antibody.

FIG. 36 is a diagram showing the result of Western blotting illustrating that pVHL expression was reduced by ZFP91 but UCP expression was increased by ZFP91.

FIG. 37 is a diagram illustrating the suppression of TNF-a mediated ZFP91 expression by the pretreatment of the NF-κB inhibitors kamebakaurin (KA), celastrol (Cel) and parthenolide (PTN) for 30 minutes and the concomitant suppression of HIF-1α expression induced under 1% partial oxygen pressure by the NF-κB inhibitors.

FIG. 38 is a diagram illustrating that kamebakaruin, known as one of NF-κB inhibitors suppressing ZFP91 expression, has an anticancer activity by inhibiting the growth of the malignant breast cancer cell line MDA-MB-435 (A) and metastasis to the lung (B) in a xenograft model.

FIG. 39 is a diagram showing the fluorescences in HEK293 cells transfected with the expression vector encoding YFP-conjugated ZFP91 protein and CFP-conjugated NIK protein:

A: photograph showing cyan by excitation;

B: photograph showing yellow protein by excitation;

C: photograph showing FRET; and

D: photograph showing the merge of A, B and C.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Screening of NF-κB Target Genes Using cDNA Microarray

As an effort to find the intracellular effector of the NF-κB inhibitor KA (kamebakaurin), cDNA microarray (Eisen M B & Brown P O, Methods Enzymol 303, 179-205, 1999) was performed to screen a gene regulated by KA. A human breast cancer cell line MDA-MB-231 cells were treated with DMSO and kamebakaurin (10 μg/ml) respectively, followed by culture for 5 hours. The cells were washed with cold PBS three times. The total RNA was extracted by using RNeasy Mini kits (Qiagen, Santa Clarita, Calif., USA), followed by 17K human cDNA microarray performed by GenomicTree Co. (KR). As a result, 333 genes up-regulated in the human breast cancer cell line MDA-MB-231 cells and 295 genes down-regulated therein were screened. To re-confirm that cDNA expression was suppressed by KA, RT-PCR with 50 genes selected among them was performed as follows.

### EXAMPLE 2

### Selection of Target Gene Candidates of Kamebakaurin by RT-PCR and Northern Blotting

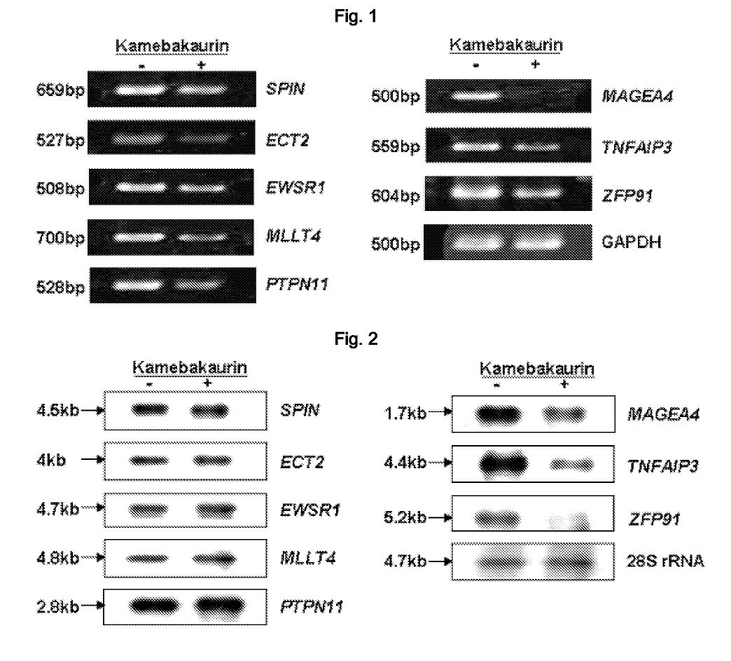

A human breast cancer cell line MDA-MB-231 cells was treated with DMSO and kamebakaurin (10 μg/ml) respectively, followed by culture for 5 hours. The cells were washed with cold PBS three times, and then total RNA was extracted by using RNeasy Mini kits (Qiagen, Santa Clarita, Calif., USA) 5 μg of the total RNA was used for the synthesis of cDNA using Invitrogen kit Access RT-PCR Kit (Promega, Madison, Wis., U.S.A.). The ZFP91 specific primer was constructed based on the nucleotide sequence of Genebank (Accession No. NM-053023). Each candidate gene specific primer was constructed based on the informed nucleotide sequences (Table 1). PCR was performed with prepared cDNA as follows: predenaturation at 95° C. for 5 minutes, denaturation at 94° C. for 1 minute, annealing at 50° C.-60° C. for 2 minutes, polymerization at 70° C. for 1 minute, 30 cycles from denaturation to polymerization. The gene-specific PCR products proceeded to electrophoresis on 1% agarose gel, followed by EtBr staining (FIG. 1).

As a result, 8 genes which were down-regulated by at least two times of the normal expression by KA were selected (PTPN11, SPIN, ECT2, EWSR1, MLLT4, MAGEA4, TNFAIP3 and ZFP91), followed by Northern blotting (FIG. 2).

TABLE 1

Primers for PCR amplification
of candidate gene

| | | |
|---|---|---|
| | Primer sequence | SEQ.ID. |
| Clone | (sense/antisense) | NO. |

| | | |
|---|---|---|
| SPIN | TAC CAC ATG GTC TCC CAG TTT | 5 |
| (Spindlin) | CAT TTG CTT CAC CAG TGC AT | 6 |
| EGT2 | AGC TTT GCA ACC CTG AGA GAG | 7 |
| (Epithelial | CCA CAA TTT TCC CAT GGT CT | 8 |
| cell | | |
| transforming | | |
| sequence | | |
| 2 oncogene) | | |
| EWSR1 | GAG CTG GAG ACT GGC AGT GT | 9 |
| (Ewing | TAG CAC CAG GAA GCT GAG GG | 10 |
| sarcoma | | |
| breakpoint | | |
| region 1) | | |
| MLLT4 | CTC AAG CAC AGC TGT CAC TCA | 11 |
| (Myeloid/ | CAA GCA AGC AAA TCC TTC CT | 12 |
| lymphoid or | | |
| mixed-lineage | | |
| leukemia; | | |
| translocated | | |
| to, 4) | | |
| PTPN11 | AAA GAA TAT GGC GTC ATG CG | 13 |
| (Protein | CGT CTG GTC CGC TAG AGA AT | 14 |
| tyrosine | | |
| phosphatase, | | |
| nonreceptor | | |
| type 11) | | |
| MAGEA4 | AAG GAG CTG GTC ACA AAG GC | 15 |
| (Melanoma | ACC CTG ACC ACA TGC TCC A | 16 |
| antigen | | |
| family A, 4) | | |
| TNFAIP3 | GTT TTC TGG TTG TTG TTG GGG | 17 |
| (Tumor | AAT ACC AGG GTA CCA TGG GAT | 18 |
| necrosis | | |
| factor, | | |
| alpha- | | |
| induced | | |
| protein 3) | | |
| ZFP91 | TCA GAG TGT TGC AGA TTT GCC | 19 |
| (Zinc finger | GGG AAA CGG CTG AGA TAG TTT | 20 |
| protein 91 | | |
| homolog) | | |

MDA-MB-231 cells were treated with DMSO and kamebakaurin (10 μg/ml), followed by culture for 5 hours. The cells were washed with cold PBS three times, and poly(A)⁺ RNA was extracted by using FastTrack 2.0 kit (Invitrogen). 2 μg of the extracted poly (A)⁺ RNA was eletrohporesed on 1% agarose-formaldehyde gel, which was transferred onto a nylon membrane (Roche, Mannheim, Germany). To investigate the expressions of selected candidate genes, the PCR product was purified by using Wizard PCR PrepsDNA Purification Systems (Promega Corporation, Madison, Wis., U.S.A.) and then used as a probe. Each probe was labeled with [α-³²P]dCTP using Rediprime™ II random prime labeling system (Amersham Biosciences). Non-incorporated ³²P was removed by using spin column (ProbeQuant™ G-50 Micro Columns; Amersham Biosciences). The prepared cDNA was used as a probe for hybridization with the membrane. After the hybridization, the membrane was washed with 2×SSC/0.1% SDS, which was exposed on X-ray film at −80° C. with intensifying screen.

It was confirmed from the Northern blotting that expression levels of MAGEA4, TNFAIP3 and ZFP91 were significantly different from the KA-non-treated control (FIG. 2). Many reports on MAGEA4 and TNFAIP3 had been made (Gillespie et al., 1999; Hillig et al., 2001), whereas ZFP91 still has a lot to be disclosed in its functions and relation to NF-κB. So, the present inventors continued to study the functions of ZFP91.

### EXAMPLE 3

### Analysis of Amino Acid Sequence and Motif Promoter Region of ZFP91

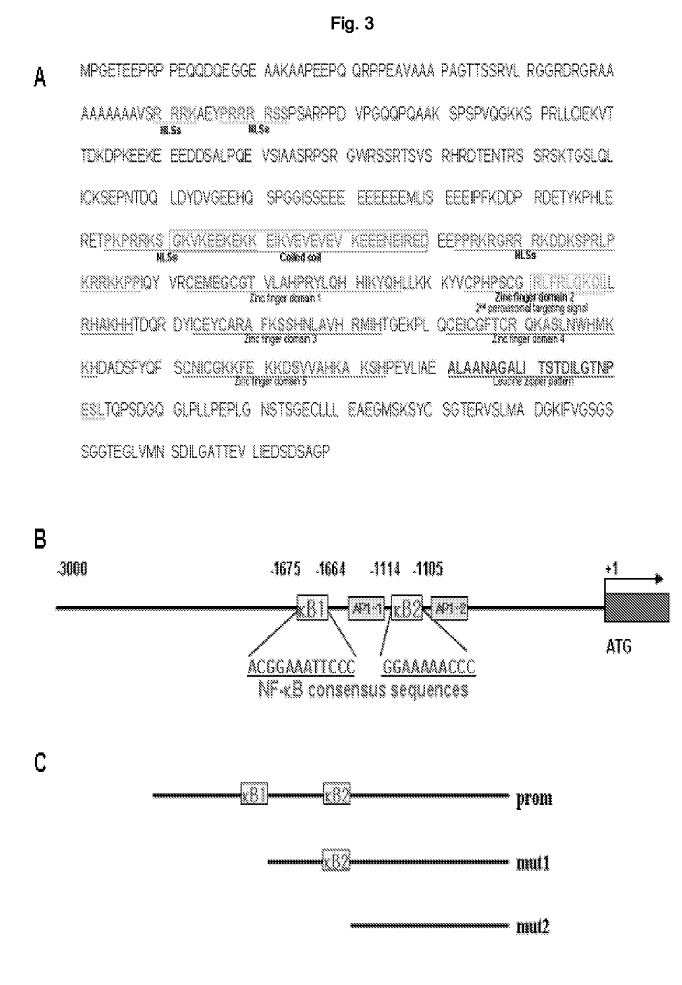

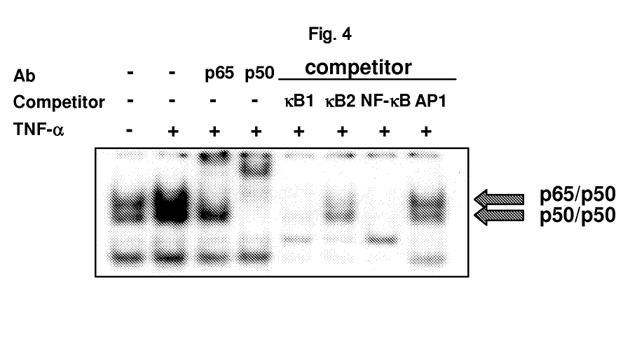

ZFP91 was confirmed to be composed of 570 amino acids and expected size of this protein was about 63 kDa (actual size in cell was confirmed to be 91 kDa; Unoki et al., Int J Oncol 22, 1217-1223, 2003). It was confirmed from the data analysis on ZFP91 that ZFP91 has 5 Zinc finger domains, one coiled coil, and leucine zipper pattern. Its 5′ region analysis revealed two NF-κB consensus sequences in −1105 and −1664 regions of 5′ upstream region (Ensembl Genome Browser; http://www.ensembl.org, MOTIF: Searching Protein and Nucleic Acid Sequence Motifs; http://motif.genome.ad.jp). It was also presumed to have four nuclear localization sequences and 87% was the chance of being in nucleus (TargetP Server v1.01; http://www.cbs.dtu.dk/services/TargetP) (FIG. 3).

### EXAMPLE 4

### Analysis of Transcription Regulation Site of ZFP91

To examine whether NF-κB was bound to two KB-binding consensus sequences of ZFP91 promoter, electrohporetic mobility shift assay (EMSA) was performed. The stomach cancer cell line SNU-638 over-expressing ZFP91 was stimulated with TNF-α for 30 minutes, followed by isolation of a nucleic extract (Lee et al., J Biol Chem 277, 18411-18420, 2002). 20 μg of the nuclear extract was reacted with anti-p65 (ReIA) antibody (Calbiochem), anti-p50-antibody (Santa Cruz), isotope-labeled KB1 sequence (acggaaattccc, SEQ. ID. NO: 21) and KB2 sequence (ggaaaaaccc, SEQ. ID. NO: 22) double strand oligonucleotides and their isotope-non-labeled sequences, followed by gel electrophoresis. The gel was transferred onto 3 mM cellulose paper (Whattman 3 mM cellulose), which was exposed on x-ray film. As a result, it was confirmed that the NF-κB bound to KB1 sequence of ZFP91 promoter was composed of p65/p50 and p50/p50. When the nuclear extracts was treated respectively with p65 and p50 antibodies, supershift band was observed and at the same time the corresponding band bound to DNA was lost. When it was forced to compete with excessive KB1 and NF-κB oligonucleotide (NF-κB) purchased from Promega, NF-κB specific bands were all lost, suggesting that KB1 was actually bound to NF-κB.

### EXAMPLE 5

### Reporter Assay with ZFP91 Promoter Mutant

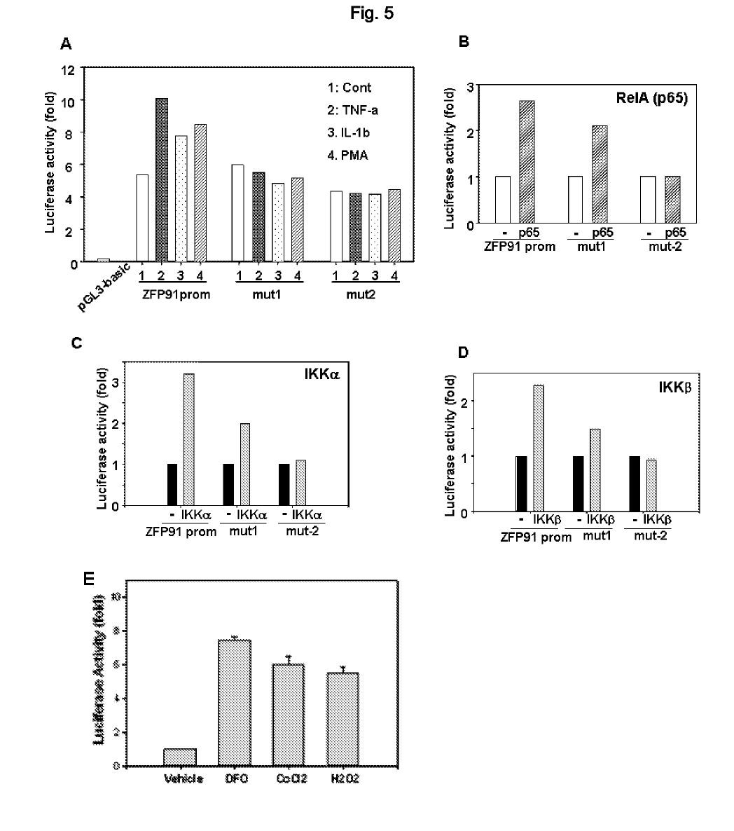

To understand the expression regulation system of ZFP91, the promoter region (−3,000) was cloned and analyzed. To analyze the cloned promoter region (FIG. 3B) precisely, deletion mutants (FIG. 3C) were generated, which were cloned into pGL3-basic vector (Promega), followed by luciferase assay to examine the interaction with NF-κB. In ZFP91 promoter containing both sequences of KB1 and KB2, the promoter activity was induced by NF-κB activators such as PMA, TNF-α and IL-1β, etc. In the meantime, in the Mut2 in which KB1 and KB2 were deleted, the promoter activity was not induced. To examine the promoter activity of each mutant, RelA, IKK a or IKKβ was over-expressed to activate NF-κB. As a result, in ZFP91 promoter containing KB1 and KB2, the promoter activity was induced by the over-expression of RelA, IKKα or IKKβ, while in the Mut2 in which KB1 and KB2 were deleted, the promoter activity was not induced, suggesting that the KB-binding region identified in ZF91 promoter, particularly KB1, plays an important role in the regulation ZFP91 expression (FIGS. 5A, B, C and D).

It was further investigated whether ZFP91 expression was induced by cellular stresses such as hypoxia or oxidative stress. First, a stomach cancer cell line was transfected with pGL3-ZFP91prom-LUC, followed by stimulation with deferoxamine (DFO), CoCl₂ and H₂O₂. Then, the promoter activity was measured. As a result, the promoter activity was significantly increased by the stimuli such as hypoxia or oxidative stress (FIG. 5E). So, it was confirmed that ZFP91 expression can be induced by various stimuli including TNF, IL-1, PMA, hypoxia, H₂O₂, etc, indicating that ZFP91 is a protein induced by stress.

### EXAMPLE 6

### NF-κB Mediated ZFP91 Expression

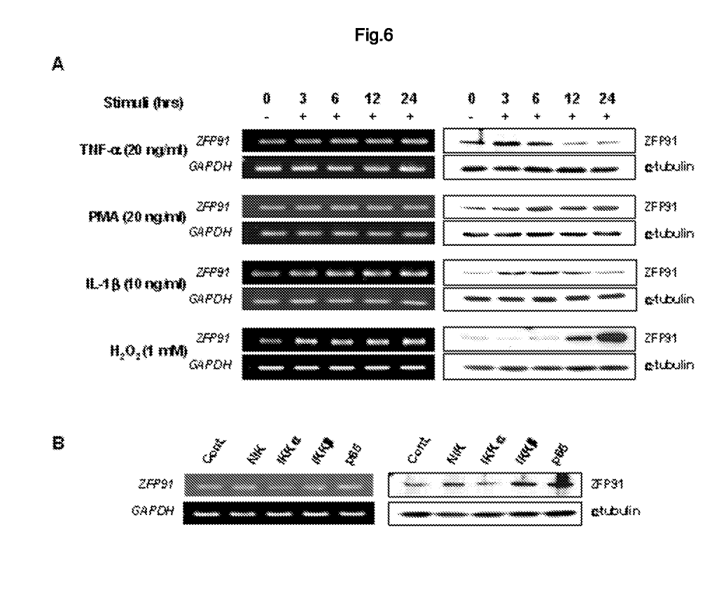

To examine whether ZFP91 expression was regulated by NF-κB, the breast cancer cell line MCF-7 was treated with the NF-κB activators, TNF-α (20 ng/ml), PMA (20 ng/ml), IL1-β (20 ng/ml) and H₂O₂ (1 mM) for 0, 3, 6, 14, and 24 hours. After 0, 3, 6, 14, and 24 hours from the treatment, poly(A)⁺ RNA was extracted, followed by Northern blotting. The probe used for Northern blotting was prepared as follows; TOPO cloning vector inserted with ZFP91 was digested with EcoRI and the insert alone was used after random prime labeling with [α-³²P]dCTP. ZFP91 expressions over the time were investigated. ZFP91 was not much increased at mRNA level, but significantly increased time-dependently at protein level. The NF-κB activators p65, IKKα, IKKβ, and NIK were expressed in MCF-7 cells. 48 hours later, ZFP91 expressions in the cells were investigated. As a result, ZFP91 was not much increased at mRNA level but significantly increased at protein level (FIG. 6). The above results indicate that ZFP91 is the gene up-regulated by NF-κB activation.

### EXAMPLE 7

### Analysis of ZFP91 Expressions in Breast Cancer Cells, Stomach Cancer Cells, Stomach Cancer Tissues, Liver Cancer Tissues and Prostatic Cancer Tissues

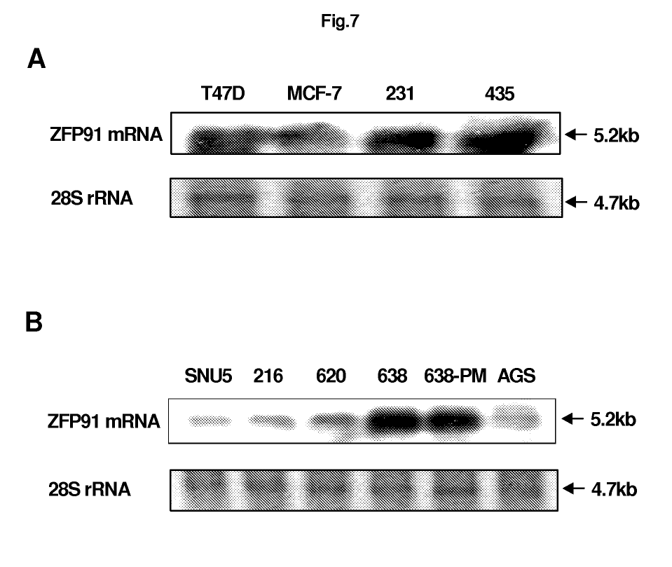

To investigate the relationship of NF-κB activation, ZFP91 expression and cancer malignancy, ZFP91 expressions in various cell lines including breast cancer cell lines and stomach cancer cell lines were compared by Northern blotting. As breast cancer cell lines, T47D and MCF-7 exhibiting low NF-κB activity and not malignant and MDA-MB-231 and MDA-MB-435 having high NF-κB activity and malignant were used. As stomach cancer cell lines, SNU-5, SNU-216, SNU-620, SNU-638, SNU-638-PM and AGS were used. The breast cancer cell lines and AGS were purchased from ATCC, USA, and the stomach cancer cell lines (SNU series) were purchased from Korean Cell Line Bank of Cancer Research Center, Seoul National University. SNU-638 PM was the cell line obtained from a tumor formed in a nude mouse transplanted with SNU-638 cells by hypodermic injection. Poly(A)⁺ RNA was extracted from each cell line, followed by Northern blotting to examine the expression of ZFP91. As a result, ZFP91 was over-expressed in the malignant breast cancer cell lines and stomach cancer cell lines having high NF-κB activity (FIG. 7).

ZFP91 mRNA expressions in normal stomach tissues and stomach cancer tissues, normal liver tissues and liver cancer tissues, normal prostatic tissues and prostatic cancer tissues were examined by in situ hybridization. For in situ hybridization, ZFP91 forward and reverse riboprobes were synthesized from human cDNA fragment inserted into pBluescriptII KS vector containing T3 and T7 promoters by using T3 and T7 RNA transcription enzymes. The normal mucus layer and invasive tissues of stomach cancer tissues were fixed in 4% paraformaldehyde solution, and then the solution was replaced with 30% sucrose solution, which stood for overnight. Frozen sections (30 μm in thickness) were prepared and fixed on the slide, which was treated with 0.4% Triton X-100. The slide was treated with protease K (25 g/ml) at room temperature for 20 minutes. The slide was put in hybridization solution (0.5 mg/ml tRNA, 20 mM Tris-HCl (pH 8.0), 2.5 mM EDTA, 1×Denhardt's solution, 0.3 M NaCl, 50% deionized formamide, 0.1% Tween 20, and 0.5 g/ml digoxigenin-labeled ZFP91 antisense or sense riboprobe), which stood at 55° C. for overnight. Then, the slide was washed with 2×SSC/50% formamide solution at 55° C. for one hour, with 11×SSC/50% formamide solution at 55° C. for 1 hour and then with 0.5×SSC/50% formamide solution at 60° C. for 1 hour. The slide was put in anti-digoxigenin alkaline phosphatase conjugated antibody (diluted 1:500, Roche) solution for overnight, followed by color development in nitroblue tetrazolium/5-bromo-4-chloro-3-indolyl-phosphate (NBT/BCIP) solution.

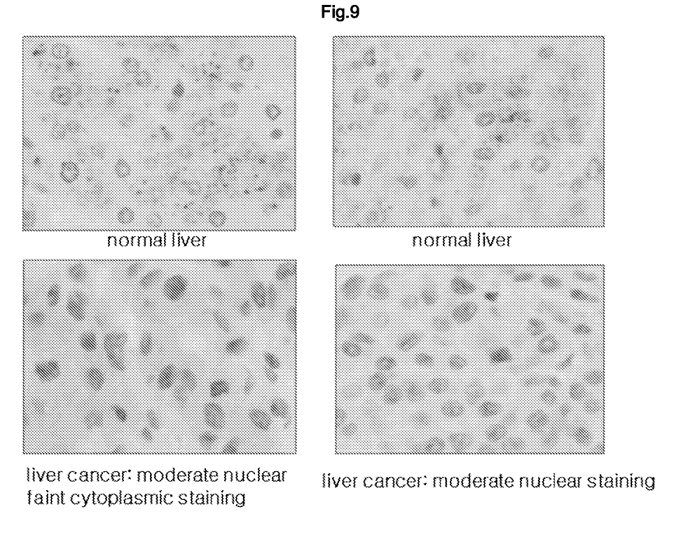

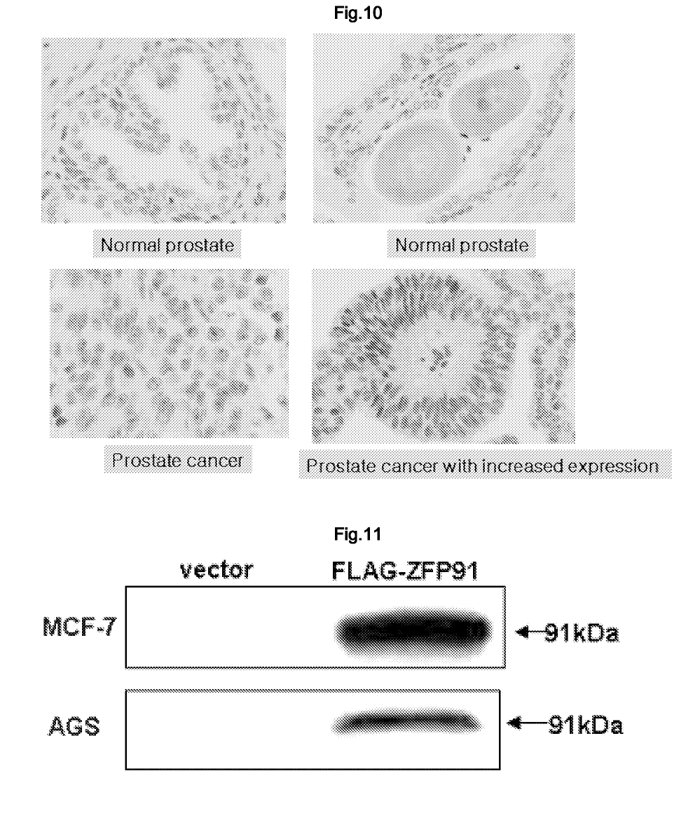

As a result, ZFP91 mRNA expression was significantly increased in stomach cancer tissues, compared with that in normal tissues (FIG. 8). In the liver cancer case, ZFP91 mRNA was also significantly up-regulated in liver cancer tissues, compared with in normal tissues (FIG. 9). ZFP91 mRNA expression was significantly increased in prostatic cancer tissues, compared with in normal tissues (FIG. 10).

### EXAMPLE 8

### Establishment of a ZFP91 Over-Expressing Breast Cancer Cell Line and a Stomach Cancer Cell Line

Among human breast cancer cell lines and stomach cancer cell lines, the breast cancer cell line MCF-7 and the stomach cancer cell line AGS exhibiting comparatively low ZFP91 expressions and being not malignant were transfected with ZFP91 to establish stable cell lines. ZFP91 was over-expressed in those cell lines to investigate the functions of the gene.

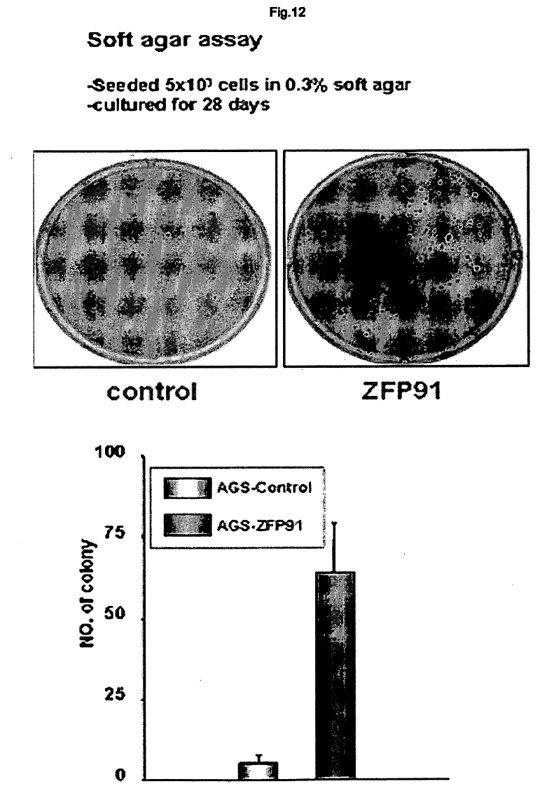

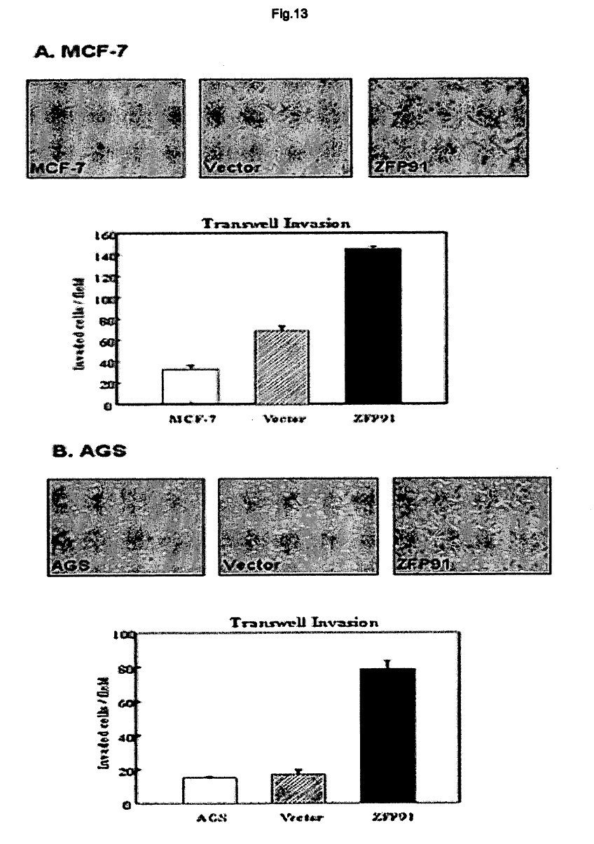

First, ZFP91 containing 5′-BamHI site and 3′-Xho I site was cloned into the suitable frame of the FLAG-labeled expression vector, pCMV-Tag2B (Stratagene). The breast cancer cell line MCF-7 and the stomach cancer cell line AGS (ATCC, USA) were transfected with the FLAG-labeled expression vectors for pCMV-Tag2B (Stratagene) and ZFP91 cloned in pCMV-Tag2B, followed by selection using G418 medium. The selected MCF-7 and AGS cells that is MCF-7-ZFP91 and AGS-ZFP91 cells transfected with ZFP91 and MCF-7-vector and AGS-vector cells transfected with pCMV-Tag2B were prepared and ZFP91 expressions in those cells were investigated by Western blotting using anti-FLAG monoclonal antibody (Sigma, USA) (FIG. 13).

As a result, 91 kDa sized ZFP91, which was bigger than expected (expected molecular weight was 63 kDa), was confirmed in those cells transfected with ZFP91, suggesting that the ZFP91 over-expressing breast cancer cell line and stomach cancer cell line were stably established.

### EXAMPLE 9

### Analysis of Anchorage-Independent Cell Growth

### Soft Agar Assay

Anchorage-independent cell growth of AGS transfected with ZFP91 was analyzed by investigating colony formation. 1% Hard-agarose and RPMI1640 serum free medium were mixed at the ratio of 1:1, resulting in 0.5% agarose, which was distributed in a 6-well plate by 2 ml per well. The mixture was hardened at 4° C. for 10 minutes and then at room temperature for 10 minutes. 1×10³ AGS cells over-expressing ZFP91 stably or not were floated in RPMI1640 containing 1% PSG and 20% serum, to which 0.3% low temperature melting agarose (GIBCO™ Invitrogen Corporation) was added and mixed, resulting in 4 ml each. The medium was loaded on the hardened agarose, which was cultured in a 37° C., 5% CO₂ incubator for 4 weeks. The cells were stained with 0.1% crystal violet in 40% methanol. The number of colonies of at least 1 mm in size was counted. As a result, ZFP91 increased colony formation of the stomach cancer cell line AGS significantly in the soft agar.

### EXAMPLE 10

### Effect of ZFP91 on Invasion of Breast Cancer Cells and Stomach Cancer Cells

To investigate whether ZFP91 was involved in invasion of cancer cells in the early stage of metastasis, the effect of ZFP91 on invasion was examined. The bottom of Modified Boyden Chamber filter (Corning Costar, Cambridge, USA) was coated with 6.5 μg of fibronectin (Roche, Mannheim, Germany), the chemoattractant, and the upper part was coated with 20 μg of matrigel (Collaborative Biomedical Products, USA). 800 μl of RPMI 1640 medium containing 10% FBS was loaded in the lower sector of the Boyden Chamber transwell. In the meantime, the breast cancer cell lines MCF-7, MCF-7-vector, and MCF-7-ZFP91 were added to 0.5% BSA RPMI1640 medium at the concentration of 3×10⁵ cells/ml, while the stomach cancer cell lines AGS, AGS-vector, and AGS-ZFP91 were distributed on the upper sector of the transwell at the concentration of 1×10⁵ cells/ml. MCF-7 cells were cultured for 4 days and AGS cells were cultured for one day. The cells which could not pass through the filter after the culture were wiped out with a cotton swab. The cells moved to the lower part of the filter were fixed with methanol. The cells were then stained with hematoxylin and eosin (Sigma). 5 microscopic fields (100×) per filter were selected randomly and cell numbers were counted.

As a result, invasion was increased in 2.1 fold for MCF-7 cells over-expressing ZFP91 and 4.6 fold for AGS cells over-expressing ZFP91 compared with respective control (FIG. 13).

### EXAMPLE 11

### Effect of ZFP91 on the Expressions of Anti-Apoptotic Proteins and Cell Cycle Inhibiting Proteins

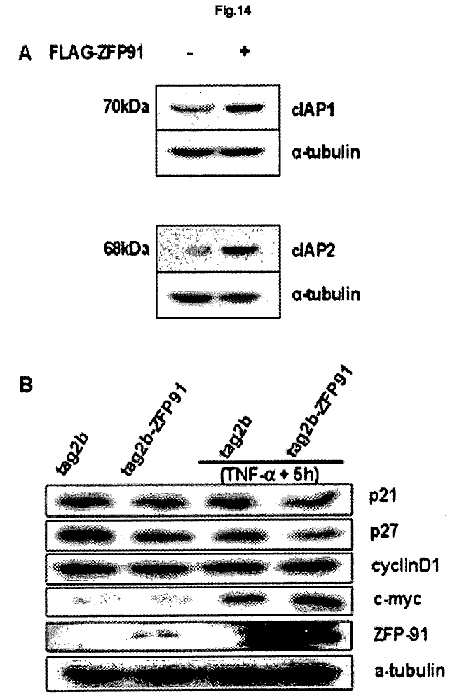

The effect of ZFP91 on the expression of some of NF-κB target genes having anti-apoptosis activity was investigated. MCF-7 cells were transfected with ZFP91 expression plasmid vector and control vector. The cells were recovered and frozen at −70° C., followed by lysis in a lysis buffer (50 mM Tris, 0.5 mM EDTA, 50 mM KCl, 10% Glycerol, 1 mM DTT, 0.5% NP-40, 0.5 mM PMSF) to prepare cell extract. The cell extract proceeded to Western blotting using anti-cIAP1 antibody, anti-cIAP2 antibody (Santa Cruz) and a-tubulin antibody (Sigma). As a result, the expressions of cIAP1 and cIAP2 were increased in cells over-expressing ZFP91 (FIG. 14B).

Next, the effect of ZFP91 on the expressions of p27(KIP1) and p21(CIP1) inhibiting CDK2 that is the kinase important in the entering to S phase in G1 which is an important phase of cell cycle involved in cell proliferation was investigated in the stomach cancer cell line AGS by western blot analysis using anti-p21 antibody, anti-p27 antibody (Santa Cruz) and a-tubulin (Sigma) antibody. As a result, the expressions of p27 and p21 in those cells were suppressed by ZFP91 (FIG. 14B).

### EXAMPLE 12

### Construction of an Antibody Using Amino Acids 91˜200 Fragments of ZFP91

cDNA encoding 91-200 amino acids of ZFP91 was inserted into BamHI-EcoRI site of pET-21a(+) vector (Novagen), resulting in the construction of pET-21-ZFP91 (91-200) vector. The amino acid sequence and nucleotide sequence covering from 91^{st} amino acid to 200^{th} amino acid are presented in the sequence list (SEQ. ID. NO: 23 and NO: 24). The pET-21-ZFP91 (91-200) vector was introduced into E. coli BL21, and protein expression was induced by 1 mM IPTG (isopropyl-β-D-thiogalactopyranoside) at 37° C. for 6 hours. The produced recombinant protein was purified with 6×His-tagged purification kit (Quiagen), resulting in the recombinant ZFP91 (91˜200) protein.

50 ug of the purified antigen protein was mixed with the equal amount of Freund's complete adjuvant (FCA, CHEMICON) in 0.85% saline, and this prepared solution was injected into hind paws and abdominal cavities of Balb/c mice (female, 6 weeks) by 50 ul each. 2-3 weeks later, the yield of antigen was reduced to 25-30 ug, which was mixed with Freund's incomplete adjuvant (FIA). The prepare solution was injected into the same spot by the same manner as described above. 4-5 days later, blood sample was taken from the tail vein of the mouse, followed by measurement of the antibody titer. If the antibody titer was high, anti-serum was obtained by centrifugation of blood collected from the abdominal arota or the heart, which was left at room temperature for 30 minutes. Serum was separated by centrifugation and stored in a refrigerator until use. Pure IgG (not binding to gel) was separated from the obtained anti-serum by using QAE-Sephadex A50 ion-exchange gel (50 mM Tris/HCL pH7.2 containing 0.1 M NaCl).

Western blotting was performed to test whether the anti-serum obtained from the mouse could recognize ZFP91. The cell extracts prepared from HEK293 cells, HT-29 cells, and U937 cells and their transfectants First, 293 cells were transfected with FLAG-labeled ZFP91 expression vector constructed in the above example, followed by cell extraction. The cell extracts were analyzed by Western blotting with mixed respectively with FLAG antibody and ZFP91 anti-serum at the ratio of 500:1. As a result, the prepared ZFP91 anti-serum could recognize ZFP91 specifically in around 90 kDa (FIG. 15).

Also ZFP91 anti-serum was efficiently recognized the ZFP91 protein expressed in various breast cancer cell lines and stomach cancer cell lines. These Western blot analysis was consistent with the mRNA expression of ZFP91 in those cells. (FIG. 15).

### EXAMPLE 13

### Effect of ZFP91 siRNA Mediated ZFP91 Expression Suppression on HIF-1 and NF-κB Activation

To investigate the effect of ZFP91 on HIF-1 activation, a human stomach cancer cell line, AGS cells and AGS cells transformed with ZFP91 were transfected with HRE dependent reporter plasmid. ZFP91 siRNA oligomers (SEQ. ID. NO: 1-NO: 4; siRNA sequences corresponding to amino terminal region ccaggtg gcattagtag tgaa, sense: r(AGG UGG CAU UAG UAG UGA A)dTdT, antisense: r(UUC ACU ACU AAU GCC ACC U)dGdG; produced by GIAGEN) were introduced into the above cell lines by using RANiFect Transfection reagent (QIAGEN, Germany).

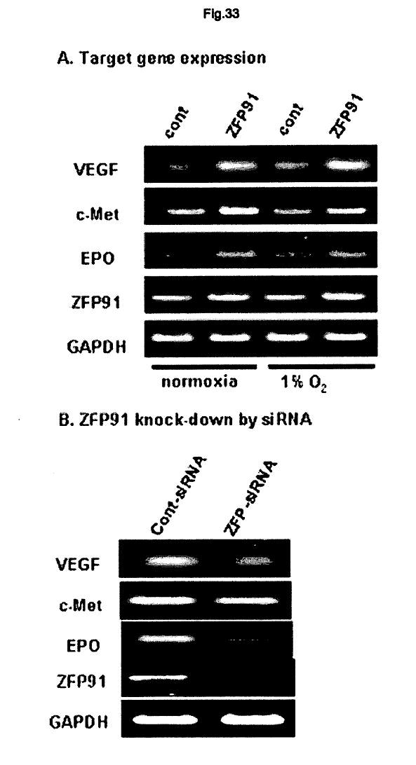

Particularly, After 24 hours incubation of 1×10⁵ cells/well distributed in a 24-well plate. siRNA oligomer (3.8 ul of SIRNA oligomer: 1 ug) was introduced into the cells with 96.2 ul of medium or EC-R buffer and 6 ul of RNAiFect reagent (Quiagen) and then the plate was kept at room temperature for 10-15 minutes. During the incubation, the medium of 24-well plate was replaced with 300 μl of fresh medium and the mixture of siRNA oligomer and RNAiFect reagent was slowly added and carefully mixed. The reaction mixture was incubated for 24 hours under normoxic condition, and then further cultured for another 24 hours in an incubator in the presence of 1% oxygen. Some of extracts of the cells proceeded to luciferase assay and HIF-1 induced luciferase activity was measured. Some of extracts of the cells proceeded to Western blotting to measure the expressions of HIF-1α and ZFP91. As a result, HIF induced luciferase activity was reduced in both cells at least 80% (FIGS. 31A and 31B). ZFP91 expression and HIF-1α expression were also reduced significantly (FIG. 31C). ZFP91 siRNA oligomer was introduced into AGS cells by the same manner as described above. mRNA expressions of the target genes such as VEGF, EPO, and cMET mediated by HIF were also investigated by RT-PCR in the presence of 1% oxygen. As a result, ZFP91 siRNA oligomer significantly reduced the expressions of these target genes (FIG. 33B).

To investigate the effect of ZFP91 on NF-κB alternative pathway, the human breast cancer cell line MDA-MB-231 was used. Particularly, ZFP91 siRNA oligomer was introduced into MDA-MB-231 cells, followed by culture in an incubator for 48 hours. Then, p52 level was measured in the cell extract. As a result, p52 level was reduced by ZFP91 siRNA oligomer (FIG. 21D).

### EXAMPLE 14

### Effect of the Suppression of ZFP91 Expression on Apoptosis

To investigate the effect of ZFP91 on apoptosis, the human breast cancer cell line MCF-7 was introduced with ZFP91 siRNA oligomer and ZFP91 expression was suppressed followed by MTT assay. Particularly, MCF7 1×10⁵ cells/well were distributed in a 24-well plate. 24 hours later, control siRNA oligomer and ZFP91 siRNA oligomer (SEQ. ID. NO: 1-NO: 4) were introduced into the cells. 48 hours later, the cells were treated with MTT reagent (Promega, USA), followed by culture in a CO₂ incubator for 3 hours. Lysis buffer (PC-12, 2% SDS, 50% DMF(pH 7.4)) was added to each well by the equal volume and mixed well to lyse the cells. The cell lysate was transferred to a 96-well plate, and Absorbance of solution was measured by OD₄₉₀ with ELISA reader.

To observe morphological changes, AGS cells were distributed to a 6-well plate, to which control siRNA oligomer or ZFP91 siRNA oligomer was introduced into the cells by the same manner as described above. 48 hours later, the cells were observed under fluorescent microscope and photographs of them were taken. Western blotting was performed to confirm the suppression of ZFP91 expression by ZFP91 siRNA oligomer.

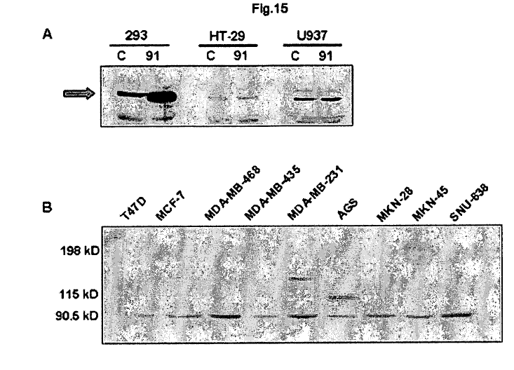

As a result, when ZFP91 expression was suppressed in cells, apoptosis was induced (FIG. 16A). siRNA oligomer was introduced into the human stomach cancer cell line AGS cells, and 48 hours later, cell morphology was observed under microscope. As a result, morphological changes of the cells were confirmed and most of the cells were floated to be dead (FIG. 16C). Also Western blot analysis confirmed that ZFP91 expression was suppressed by ZFP91 siRNA oligomer (FIG. 16B).

### EXAMPLE 15

### Effect of ZFP91 on Tumorigenesis

To investigate the effect of ZFP91 on tumorigenesis, the human stomach cancer cell line MKN45 cells with or without ZFP91 over-expression was hypodermically injected into nude mice (female) at 5 weeks (Charles River Laboratory, Wilminton, USA). Tumorigenesis was observed for 4 weeks.

Particularly, the human stomach cancer cell line MKN45 cells transfected with a vector or ZFP91 was treated with trypsin-EDTA and separated from the culture dish. The cells were washed with sterilized PBS twice. The cells were suspended in sterilized saline at the concentration of 1×10⁷ cells/0.1 ml. The cells were hypodermically injected into 6 nude mice without thymus at the concentration of 1×10⁷. After 2 weeks from the injection, tumor development was observed and tumor size was measured every 5 days. After the measurement of tumor development and the size, blood was taken from the eyes of the mice. VEGF level in the blood was quantified by Sandwich ELISA using Quantikine human VEGF kit (R&D, USA).

As a result, tumorigenesis was confirmed in 5 out of 6 nude mice injected with MKN45 cells without ZFP91 over-expression. In the meantime, tumorigenesis was confirmed 6 out of 6 nude mice transplanted with MKN45 cells with ZFP91 over-expression. The size of the tumor developed in nude mice injected with ZFP91 over-expressing cells was much larger than that of the nude mice injected with MKN45 cells with control vector ZFP91 and the tumor growth rate was also much faster in the mice injected with ZFP91 over-expressing cells (FIGS. 17A and B). Serum was also obtained from the eyes of the nude mice to measure VEGF level in the serum using Quantikine VEGF kit (R&D). As a result, VEGF level in the nude mice injected with ZFP91 over-expressing MKN45 cells was much higher than that of the control (FIG. 17C). It was also observed that many blood vessels were formed in the tumor induced in ZFP91 over-expressing MKN45 cells. The above results confirmed that ZFP91 inhibits apoptosis, induces tumor growth and promotes angiogenesis.

### EXAMPLE 16

### Analysis of ZFP91's Effect on NF-κB Activation

In this invention, it was observed that ZFP91 could induce cancer malignancy by activation of NF-κB signaling pathway. Thereafter, whether or not this phenomenon depended on ZFP91 expression was investigated. ZFP91 was over-expressed at different concentrations in HEK293 cells and then NF-κB activity therein was measured by luciferase assay.

Particularly, 1×10⁵ HEK 293 cells were distributed in a 12-well culture dish, followed by culture for 12 hours. 0.7˜1.5 μg of plasmid DNA was introduced into the cells by using LIPOFECTAMINE reagent (Invitrogen, USA), followed by further culture for 24 hours. The cells were cultured in serum-free medium for 3 hours and then stimulated with various stimuli for 3-12 hours. The cells were lysed in 1× passive lysis buffer (Promega) and the luciferase activity was measured by Microlumat Plus luminometer (EG&G Berthold, Bad Wildbad, Germany) using dual luciferase assay system (Promega, USA). The tesults was normalized to the renilla luciferase activity.

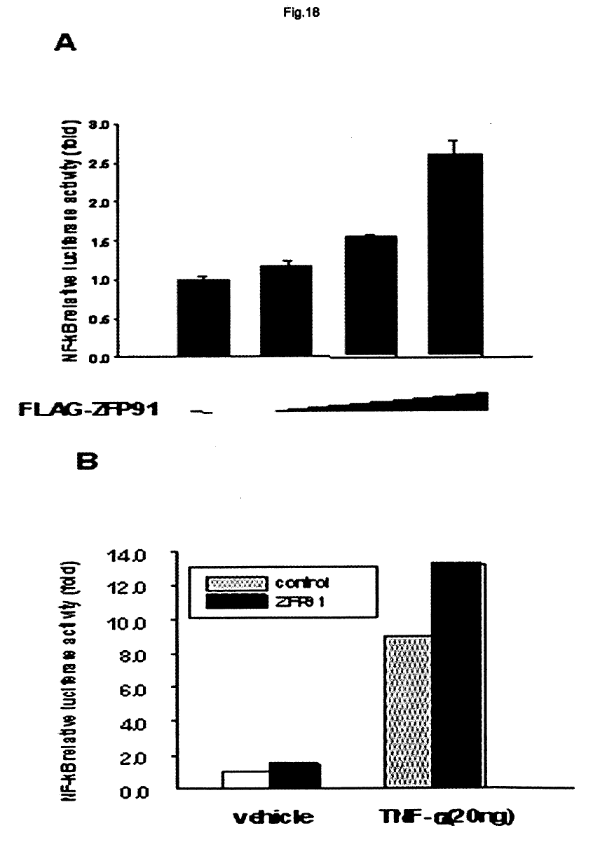

As a result, NF-κB activity was increased ZFP91 dose dependently (FIG. 18A), which was more significant when TNF-α (20 ng/ml) was co-treated (FIG. 18B). The above results indicate that ZFP91 affects NF-κB.

### EXAMPLE 17

### Effect of ZFP91 on the Transcription Activity of p65

P65 is the representative NF-κB subunit and its activation is important in NF-κB activity. To examine the effect of ZFP91 on the activation of p65, the NF-κB transcription factor, p65 and its mutants with transcription activation domain 1(TA1 domain: p65 amino acids 521-551 including Ser⁵³⁶) were expressed in HEK293 cells together with ZFP91 expression plasmid. Then, it was investigated by luciferase assay to see whether or not the transcription activity was increased by ZFP91. cDNA encoding TA1 region (amino acids 521˜551) of human p65 protein was amplified by PCR and the PCR product was cloned into pFA-CMV vector (Stratagene), resulting in the construction of pFA-CMV-p65 (521˜551) plasmid. The effect of ZFP91 on the transcription activity of TA1 region of p65 was investigated by using reporter plasmid pFR-Luc (Stratagene).

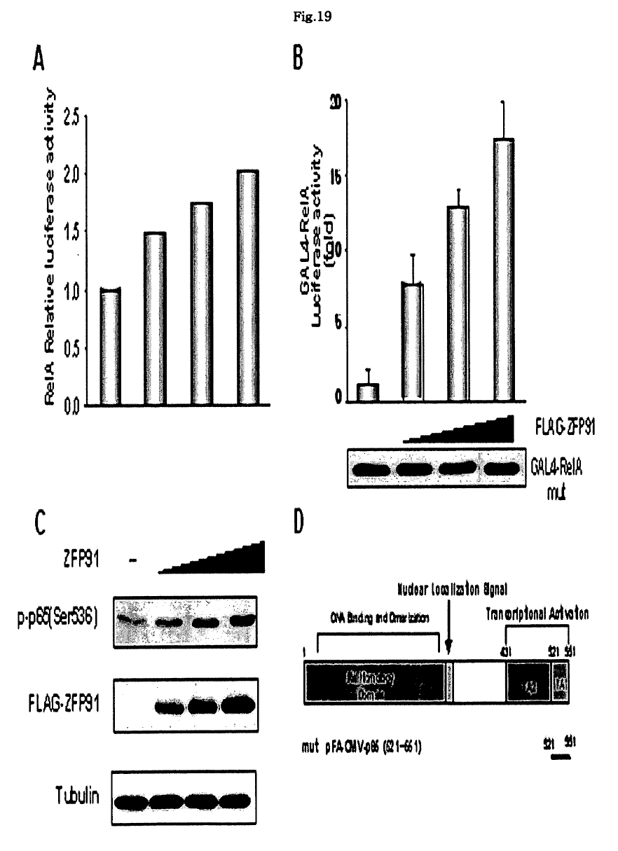

As a result, the transcription activity of p65 was increased ZFP91 dose dependently. In particular, the transcription activity of p65 mutant containing the transcription activation domain 1 (TA1) only was significantly increased (FIG. 19C). Phosphorylation of p65 Ser536 which has been know to be important factor for p65 activation was investigated using anti-phospho (Ser536) p65 antibody (Cell Signaling) (FIG. 19A). As a result, p65 phosphorylation was increased ZFP91 dose-dependently (FIG. 19C).

### EXAMPLE 18

### Effect of ZFP91 on the Activation of NF-κB Signaling Induced by Various Activators

To examine how ZFP91 could increase NF-κB, p65 transcription activity, ZFP91 was co-expressed with various NF-κB activators (NIK, TRAF2, IKKα and IKKβ) in HEK293 cells. 24 hours later, luciferase assay was performed to compare the NF-κB activities. 1×10⁵ HEK293 cells were distributed in a 12-well culture dish, followed by culture for 12 hours. 0.2 μg of NF-κB luciferase reporter plasmid DNA and pFR-Luciferase were introduced into the cells using LIPOFECTAMINE reagent (Invitrogen). 8-12 hours later, the molecules inducing NF-κB activation were expressed, followed by further culture for 8-12 hours. The cells were lysed in 1× passive lysis buffer and the luciferase activity was measured by Microlumat Plus luminometer (EG&G Berthold, Bad Wildbad, Germany) using dual luciferase assay system (Promega, USA). Intracellular introduction efficiency of the plasmid DNA was corrected by measuring the renilla luciferase activity.

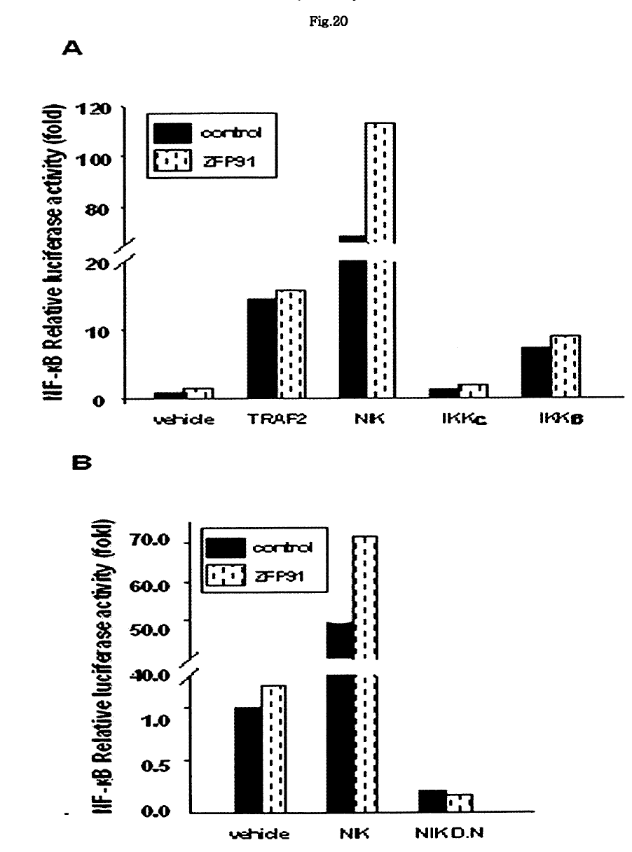

As a result, all the above activators increased NF-κB activity and when they were co-expressed with ZFP91, the increase was more significant (FIG. 20A). In particular, ZFP91 affected NIK mediated NF-κB activation most significantly. To examine the effect of ZFP91 on NIK, NIK dominant negative form was co-expressed with ZFP91, and then NF-κB activation was measured. As a result, ZFP91 mediated NF-κB activation was not observed when NIK dominant negative form was co-expressed with ZFP91 (FIG. 20B), indicating that ZFP91 plays an important role in NIK mediated NF-κB activation.

### EXAMPLE 19

### Effect of ZFP91 on the Molecules Inducing NIK Mediated NF-κB Activation

The effect of ZFP91 on NIK mediated NF-κB activation pathway was examined. NIK alone was over-expressed or NIK was co-expressed with ZFP91 in HEK 293 cells, followed by investigation of the expressions of NIK downstream molecules IKKα and IKKβ, p65 phosphorylation, IκBA degradation, processing of p100 digestion to p52 by Western blotting. As a result, NIK mediated p65 and IKKα phosphorylation was increased when NIK was co-expressed with ZFP91. Also, ZFP91 increased phosphorylation of p38 MAP kinase known to induce p65 phosphorylation. ZFP91 induced p100 digestion so that it increased p52 production. However, ZFP91 neither affected NIK mediated IKKβ phosphorylation nor induced IκBα degradation (FIG. 21). The above results indicate that ZFP91 significantly increases in phosphorylation of proteins involved in the NIK mediated NF-κB activation pathway, in particular increases in the processing of p100 to p52 which is important in the activation of NF-κB alternative pathway. The level of p52 in the malignant breast cancer cell line MDA-MB-231 was increased. In the MDA-MB-231 cells transfected with siRNA, p52 level was reduced more than p100 level (FIG. 21D).

The above results indicate that ZFP91 is an important molecule affecting NIK mediated NF-κB alternative pathway.

### EXAMPLE 20

### Confirmation of Interaction of ZFP91 with NIK and TRAF2

To investigate how ZFP91 induced NIK activation, NIK and ZFP91 were over-expressed in HEK293 cells, followed by co-immunoprecipitation to investigate their interaction. HEK293 cells were transfected with myc-NIK and FLAG-ZFP91 or HA-TRAF2 and FALG-ZFP91. The cells were harvested and added with denatured lysis buffer (50 mM Tris, 1% SDS, 4 M Urea), followed by lysis using ultrasonicator. The cell lysate was added with anti-FLAG, anti-NIK or anti-HA antibody, followed by immuno-precipitation at room temperature. The precipitate proceeded to Western blotting using anti-FLAG, anti-HA and anti-NIK antibodies. Some of the cell lysate was taken before the immuno-precipitation, which proceeded to Western blotting using mouse anti-FLAG antibody, anti-HA antibody, anti-NIK antibody, and anti-myc antibody. As a result, ZFP91 was confirmed to bind to NIK.

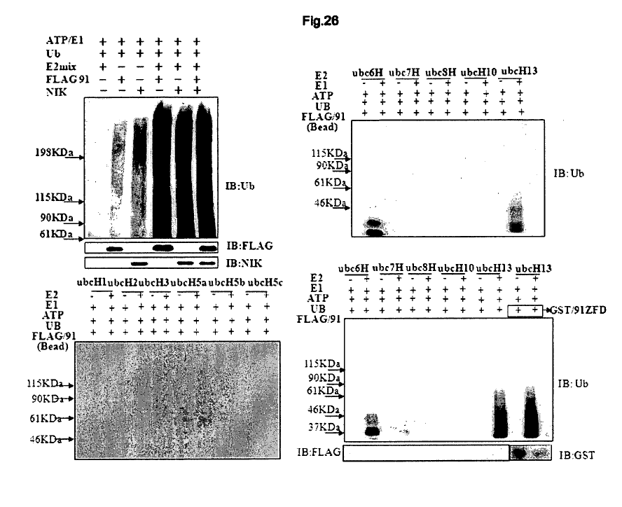

It has been known that NIK regulates the activation of NF-kB alternative pathway via its interaction with TRAF2. So, interaction between ZFP91 and TRAF2 was also investigated by IP. As a result, it was confirmed that ZFP91 also interacted with TRAF2 (FIG. 22A). To investigate whether or not these three molecules, ZFP91, NIK, and TRAF2, formed a complex, all three were over-expressed in HEK293 cells, followed by immuno-precipitation with ZFP91 to examine ZFP91 binding to NIK and TRAF2. As a result, it was confirmed that these three molecules formed a complex (FIG. 22B). Next, which domain of ZFP91 interacted with NIK was investigated. To do so, ZFP91 mutants were co-expressed with NIK, followed by immuno-precipitation. As a result, NIK was bound to the ZFP91 mutant containing Zinc finger domains but not bound to the mutant without Zinc finger domains (FIG. 23A). The above results indicate that it is the Zinc finger domains that play an important role in binding of ZFP91 with NIK.

Then, which part of NIK interacted with ZFP91 was also investigated. To do so, NIK mutants were co-expressed with ZFP91, followed by immuno-precipitation. As a result, it was confirmed that the NIK mutant containing kinase domain was bound to ZFP91 (FIG. 24A).

It was also investigated whether or not ZFP91 induced poly-ubiquitination of NIK protein. HEK293 cells were transfected with myc-NIK only, myc-NIK together with HA-Ubiquitin, or myc-NIK together with HA-Ubiquitin and Flag-ZFP91. Those cells were cultured and the cell extracts were immuno-precipitated using anti-NIK antibody by the same manner as described above, followed by Western blotting using mouse anti-HA-Ub antibody, anti NIK antibody and mouse anti myc antibody. As a result, poly-ubiquitination of myc-NIK by ZFP91 was confirmed (FIG. 25A).

Some of cell lysate was taken to measure expression of input proteins before the immuno-precipitation. To examine how NIK ubiquitination was regulated by ZFP91 in ZFP91 deletion mutant, myc-NIK was introduced into HEK293 cells with full leng of ZFP91, HA-Ubiquitin, HA-ubiquitin and FLAG ZFP91, or HA-Ubiquitin and FLAG ZFP91 mutant deletd zinc finger domains. Those cells were cultured and followed by immuno-precipitation and western blotting by the same manner as described above. As a result, poly-ubiquitination of myc-NIK was observed only in the cell group introduced with full length of ZFP91 (FIG. 25-B). The above results indicate that ZFP91 has an activity to induce poly-ubiquitination of NIK protein and ZFP91 induces NIK stabilization and activation by inducing such ubiquitination.

### EXAMPLE 21

### Analysis of In Vitro Ubiquitination

It was investigated whether or not ZFP91 and NIK ubiquitination observed in 293 cells could be induced in vitro by using ZFP91 protein isolated by using FLAG beads, ZFP91 C-term protein purified from E. coli and ubiquitination kit.

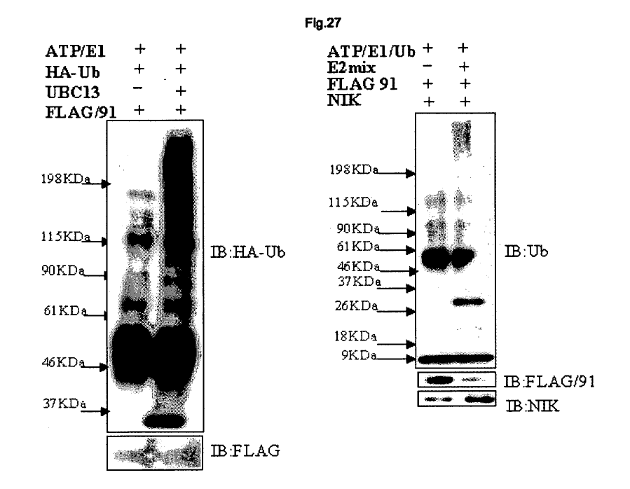

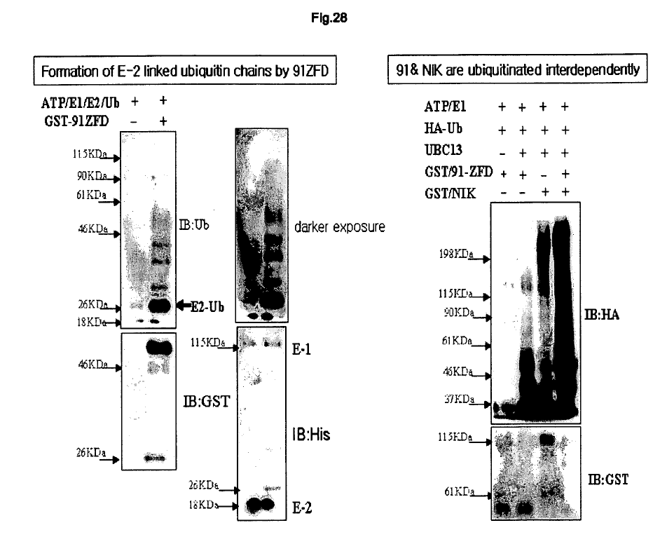

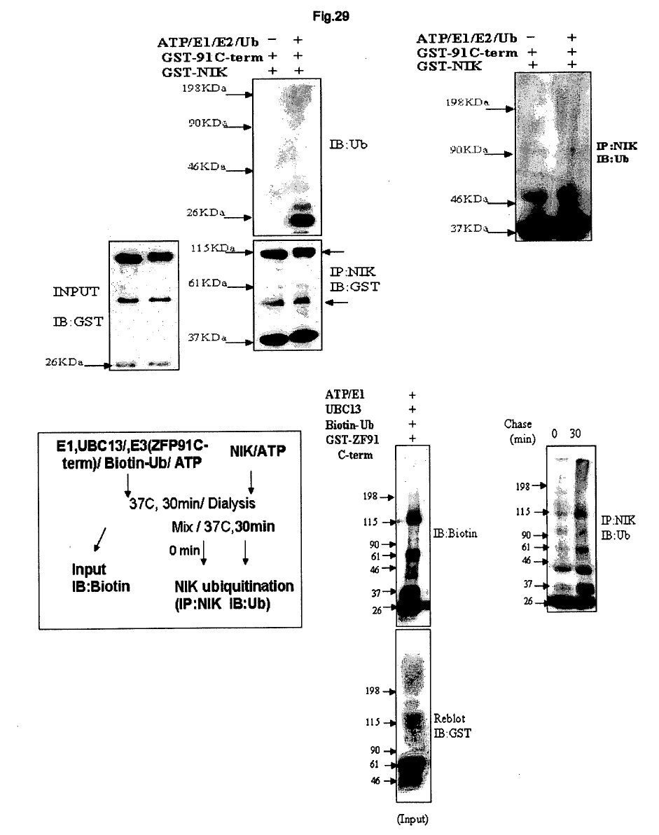

As a result, ZFP91 has been confirmed to be the protein that is auto-ubiquitinated Ubc13-depenedently and has an ubiquitin protein ligase (E-3) ligase activity. C-term protein having zinc finger domains has also been confirmed to be auto-ubiquitinated by Ubc 13. Particularly, ubiquitinated protein bands strongly showed in low molecular weight area but weakly in high molecular weight area, suggesting that there are ubiquitin chains assembled in E-2 enzyme. The ubiquitination was potentiated by the addition of NIK (FIGS. 26-29).

### EXAMPLE 22

### HIF-1 Expression by ZFP1

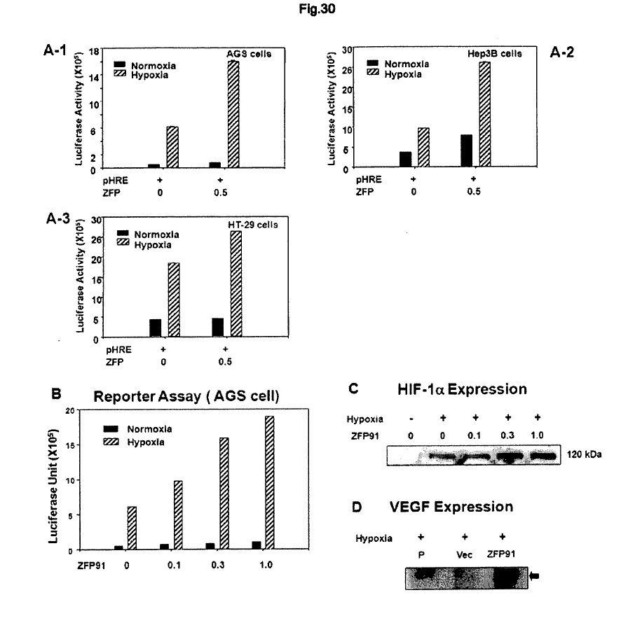

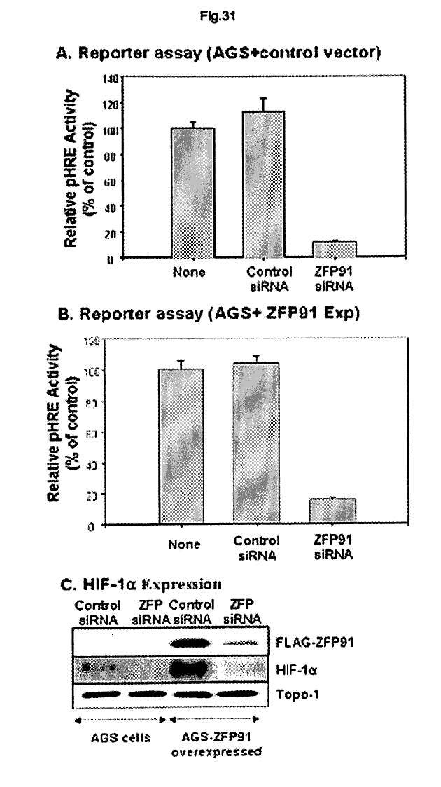

To investigate whether or not ZFP91 could increase the expression and stability of HIF-1α, reporter assay using reporter vector pHRE was performed. Various cancer cell lines including stomach cancer (AGS), colon cancer (HT-29), and liver cancer (Hep3B) were transfected with pHRE plasmid and ZFP91 expression plasmid pCMV-Tag2B-ZFP91 or control plasmid pCMV-Tag2B, followed by measuring HIF-1 activity under different oxygen concentrations (1% O₂, 20% O₂). As a result, ZFP91 significantly increased HIF-1 activity (FIG. 30-A).

The effect of ZFP91 on HIF-1 activation was investigated in the stomach cancer cell line AGS. The AGS cells were transfected with pCMV-Tag2B-ZFP91 at different concentrations. HIF activity was measured by using pHRE. As a result, the HIF activity was increased ZFP91 dose-dependently (FIG. 30-B). It was confirmed by Western blotting that HIF-1α protein level in the cell lysate was elevated (FIG. 30-C). Also Northern blotting confirmed increase of HIF-1 target gene VEGF mRNA expression (FIG. 30-D).

The effect of ZFP91 on the expression and activation of HIF-1α was examined by using siRNA. The AGS cells and the AGS-ZFP cells over-expressing ZFP91 were respectively transfected with ZFP91 siRNA. As a result, ZFP91 expression was significantly reduced by siRNA and so were HIF-1 activity and HIF-1α protein expression (FIG. 31).

HIF-1α protein up-regulation under hypoxia was time-dependent, which was more significant in the presence of the proteasome inhibitor MG-132 under normoxic condition. However, even in the absence of MG-132, HIF-1α protein expression was detected from 6 hours later under normoxic condition (FIG. 32-B).

### EXAMPLE 23

### Expression of HIF-1 Target Gene by ZFP91

The effects of ZFP91 on the expression of HIF-1α target genes were compared between AGS cells and AGS-ZFP cells over-expressing ZFP91. The effect of siRNA thereon was also investigated. AGS cells and AGS(ZFP91) cells were cultured respectively under normoxic condition and under 1% pO₂ condition. Total RNA was extracted from the cells by using RNeasy Mini kit (Qiagen). 5 μg of the total RNA was used for RT-PCR using Invitrogen kit Access RT-PCR kit.

As a result, the expression of HIF-1α target gene mRNA was increased in AGS(ZFP91) cells both under normoxic condition and under 1% pO₂ condition, compared with in AGS cells (FIG. 33-A). The association of these increases in mRNAs of HIF-1 target genes with the ZFP91 mRNA expression was confirmed by RT-PCR with the cells transfected with control siRNA or ZFP91 siRNA (FIG. 33-B).

### EXAMPLE 24

### ZFP91-VHL-HIF Interaction and Ubiquitination

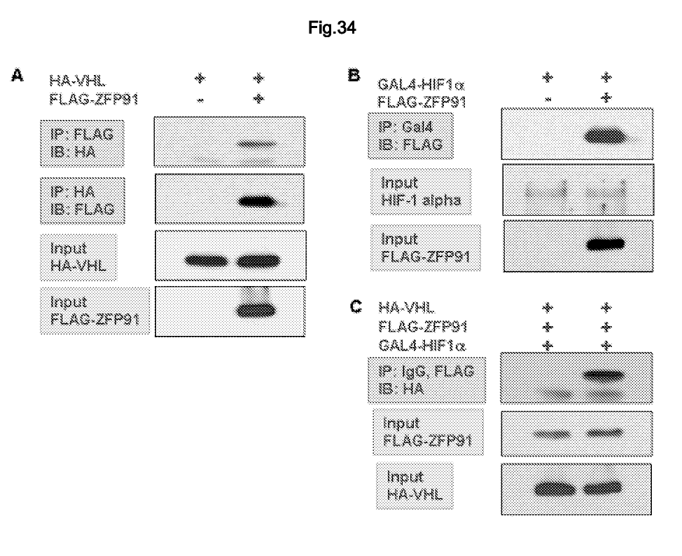

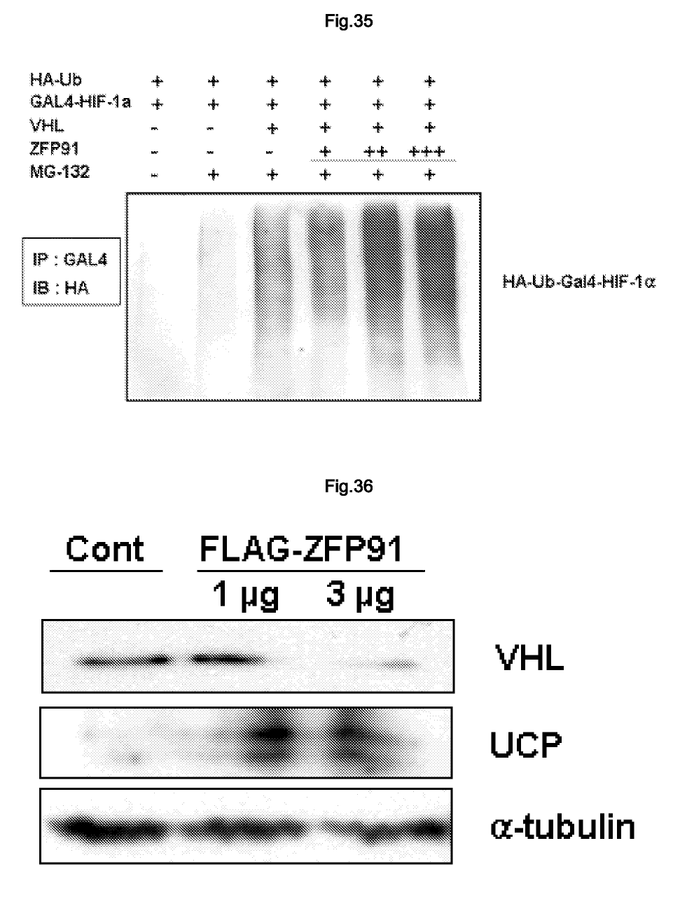

To investigate the effect of ZFP91 on HIF-1α activation, ZFP91, VHL and HIF-1α were over-expressed in HEK293 cells, followed by co-immunoprecipitation to investigate their interaction. HEK293T cells were transfected with the combination of FLAG-ZFP91, HA-VHL and GAL4-HIF-1α, followed by culture. The cell lysate proceeded to immuno-precipitation and Western blotting using each corresponding antibody by the same manner as described above. As a result, ZFP91-VHL interaction (FIG. 34-A), ZFP91—HIF-1α interaction (FIG. 34-B) and ZFP91-VHL interaction (FIG. 34-C) were confirmed. Next, it was investigated weather ZFP91 induced multiple ubiquitination of HIF-1α as it did in NIK. The combination of HA-Ub, GAL4-HIF-1α and VHL under absence or presensce increasing amount of ZFP91 DNA was expressed in HEK293 cells in the presence of the proteasome inhibitor MG132, followed by immuno-precipitation using anti-GAL4 antibody. Western blotting was performed using anti-HA antibody. As a result, it was confirmed that ZFP91 increased HIF-1α ubiquitination (FIG. 35).

### EXAMPLE 25

### Regulation of VHL and UCP Expressions by ZFP91

MCF-7 cells were transfected with ZFP91 expression plasmid using Lipofectamine plus reagent (Invitrogen), followed by culture for 48 hours. The cells were recovered and frozen at −70° C. The frozen cells were lysed in a lysis buffer (50 mM Tris, 0.5 mM EDTA, 50 mM KCl, 10% Glycerol, 1 mM DTT, 0.5% NP-40, 0.5 mM PMSF). Western blotting was performed using anti-VHL antibody (BD Sciences), anti-UCP antibody (Dr. Im, Dong-Soo; KRIBB), and tubulin antibody. As s result, in ZFP91 over-expressing cells, pVHL expression was reduced ZFP91 dose-dependently, whereas UCP expression was increased (FIG. 36).

### EXAMPLE 26

### Suppression of ZFP91 and HIF-1α Expressions by NF-kB Inhibitors

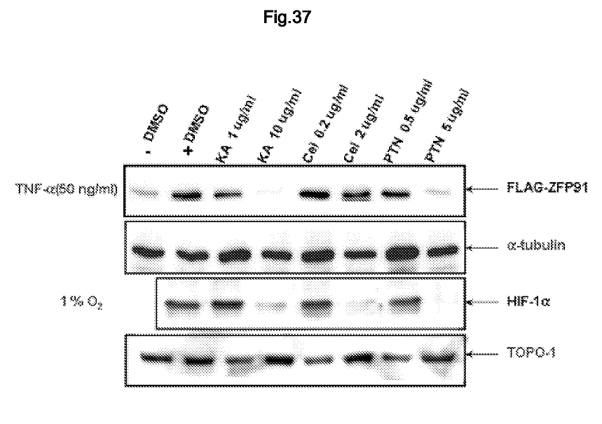

FLAG-ZFP91 expressing AGS cells were pre-treated with the NF-κB inhibitors, kamebakaurin (KA), celastrol (Cel), parthenolide (PTN), for 30 minutes. TNF-α mediated ZFP91 expression was investigated by Western blotting using anti-ZFP91 antibody. As a result, the above three compounds all suppressed TNF-α mediated ZFP91 expression. It was confirmed that the treatment of NF-κB inhibitors such as kamebakaurin (KA), celastrol (Cel) and parthenolide (PTN) to AGS cells expressing ZFP91 suppressed HIF-1α expression induced under 1% pO₂ condition (FIG. 37).

### EXAMPLE 27

### Anticancer Effect of Kamebakaurin by Inhibiting ZFP91 Expression

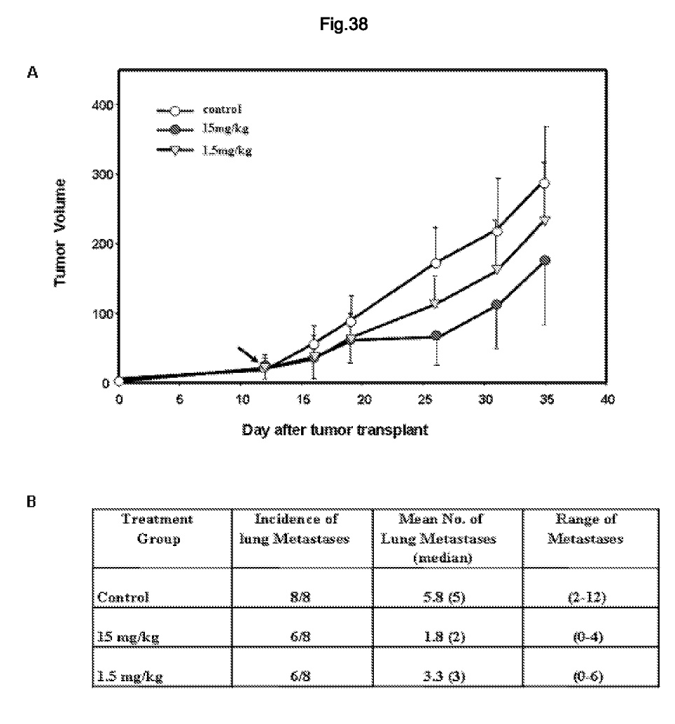

The human breast cancer cell line MDA-MB-435 was transplanted in the glandular fatty tissues of nude mice (Charles River Laboratory, Wilmington, Mass., USA) at 5 weeks (1×10⁶ cells/mouse). 12 days later, the nude mice having tumor were grouped by 8. Kamebakaurin dissolved in 0.5% Tween 80 was administered into the abdominal cavity every other day at the concentrations of 1.5 and 15 mg/kg 12 times in total. The tumor size was measured for 23 days after the transplantation. The tumor size was calculated by “width 2 (square)×length 2(square)/2=tumor volume (mm³)”. As a result, kamebakaurin significantly inhibited tumor growth at the concentration of 15 mg/kg (FIG. 38-A).

After 35 days from the transplantation, the lung of the mouse was excised and washed with water and fixed in Bouin's solution (SIGMA). The colonies metastasized to the surface of the lung (>diameter 2 mm) were counted under microscope and the mean values are shown in Table 2.

As a result, the colonies metastasized to the lung in the group treated with KA (kamebakaurin) suspended in 0.5% Tween 80 were 1.8, while the colonies metastasized to the lung in the group treated with only 0.5% Tween 80 were 5.8, suggesting that KA significantly inhibited tumor growth and metastasis to the lung (FIG. 38-B).

The above results indicate that kamebakaurin suppressing ZFP91 expression can inhibit the tumor cell growth and metastasis in mouse tumor models. Therefore, the inhibition of ZFP91 functions results in the inhibition of tumor cell growth and metastasis, suggesting that ZFP91 can be effectively used as a target molecule for the treatment of cancer.

TABLE 2
Inhibition effect of kamebakaurin on metastasis

| | | Mean number | Minimum |
|---|---|---|---|
| | Frequency | of metastasis | metastasis |
| | (metastatic | in lung | number-maximum |
| Experimental | mice/total | (median | metastasis |
| Group | mice) | value) | number |
| Control Group | 8/8 | 5.8 (5) | (2-12) |
| 15 mg/kg | 6/8 | 1.8 (2) | (0-4) |
| 1.5 mg/kg | 6/8 | 3.3 (3) | (0-6) |

### EXAMPLE 28

### Screening of ZFP91 Expression Suppressing Compounds

To screen a compound capable of suppressing ZFP91 expression, the suppression of ZFP91 and HIF expressions by HIF inhibitors and NF-κB inhibitors were investigated. Precisely, the cells were treated with these compounds by effective dosage and then HIF-1α expression was examined under 1% pO₂ condition and the effect of TNFα on the suppression of ZFP91 expression was examined by Western blotting.

To establish a screening system of ZFP91 inhibitor, plasmids in which ZFP91 was labeled with YFP and NIK was labeled with CFP were constructed. Then, interactions between these fluorescent proteins and their expressions were observed in the presence of TRAF2. Particularly, ZFP91 full length DNA was prepared to contain HindIII site at 5′ (N-term) and Kpn I site at 3′ (C-term) by PCR cloning, which was inserted into HindIII and KpnI site of pEYFP-C1 vector (BD Bioscience), resulting in the construction of yellow fluorescence labeled ZFP91 expression vector.

NIK full length DNA was prepared to contain NotI sites at 5′ and 3′, which was inserted into NotI site of pCMV-ECFP vector (BD Bioscience) by PCR cloning, followed by nucleotide sequencing. As a result, the expression vector in which blue fluorescence, CFP, was conjugated to NIK C-term was constructed.

HEK293 cells were transfected with the above vector. 20 hours later, the cells were observed under fluorescent microscope to investigate FRET (fluorescence resonance energy transfer) caused by the interaction between the two proteins.

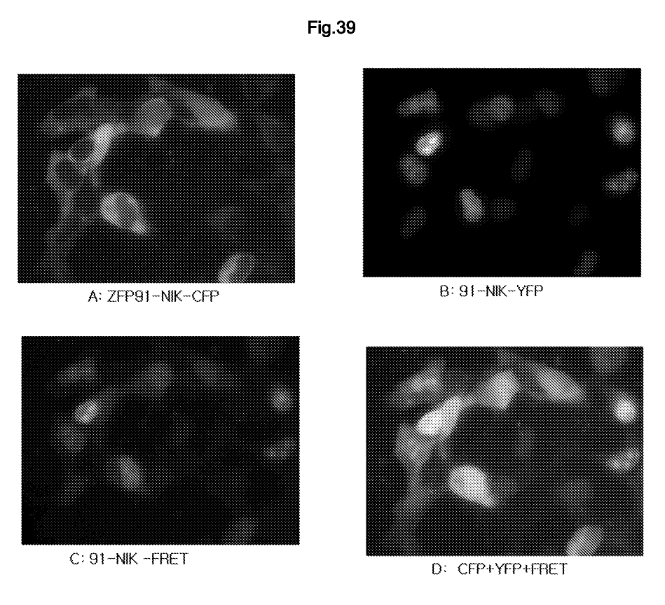

As a result, the compound suppressing ZFP91 expression inhibited HIF-1α protein expression as well (FIG. 39). Therefore, the screening of a compound suppressing expression leads the way to the development of a leading compound for a novel anticancer agent.

1. A method for inhibiting cancer the step of administering the pharmaceutically effective dose of a ZFP91 (Zinc Finger Protein 91) inhibitor to a subject with cancer.

2. The method for inhibiting cancer according to claim 1, wherein the cancer is a solid tumor.

3. The method for inhibiting cancer according to claim 1, wherein the ZFP91 inhibitor is selected from the group consisting of an antisense oligonucleotide binding complementarily to ZFP91 mRNA, a ZFP91 gene specific siRNA, an inactive ZFP91 like protein or its fragment, a ZFP91 binding peptide, a ZFP91 specific antibody, a compound specifically binding to ZFP91 mRNA to inhibit its transcription or translation, an NFκB inhibitor, and a compound binding specifically to ZFP91 protein to inhibit ZFP91 functions.

4. The method for inhibiting cancer according to claim 3, wherein the siRNA is represented by SEQ. ID. NO: 1.

5. A method for screening an anti-angiogenic agent candidate comprising:
measuring expression or activity of ZFP91 or expression of a gene regulated by ZFP91; and
selecting a sample compound inhibiting the expression or activity of ZFP91 or expression of a gene regulated by ZFP91 compared to that in the absence of the compound.

6. A method for screening an angiogenic stimulator comprising:
measuring expression or activity of ZFP91 or expression of a gene regulated by ZFP91; and
selecting a sample compound enhancing expression or activity of ZFP91 or expression of a gene regulated by the ZFP91 compared to that in the absence of the compound.

7. The method according to claim 5, wherein the expression or activity of ZFP91 is measure by the following steps:
1) contacting a sample compound with one selected from the group consisting of
i) a ZFP91 protein,
ii) a cell line transfected with an expression vector containing a reporter gene operably linked to the downstream of response region ZFP91,
iii) a cell line transfected with an expression vector containing a reporter gene construct comprising an NFκB consensus element operably lined to the reporter gene, and
iv) a ZFP91 protein and NIK or TRAF2;
2) measuring at least one aspect selected from the group consisting of
i) binding activity between ZFP91 and the sample compound when the ZFP91 protein is contacted with the sample compound,
ii) expression of the reporter gene when the transfected cell line is contacted with the sample compound, and
iii) binding activity between the ZFP91 protein and the NIK or the TRAF2 or ubiquitinylation of the NIK or the TRAF2 when the ZFP91 protein and the NIK or the TRAF2 are contacted with the sample compound; and
3) selecting the compound inhibiting at least one aspect of step 2) as compared to that in the absence of the compound.

8. (canceled)

9. The method according to claim 6, wherein the expression or activity of ZFP91 is measure by the following steps:
1) contacting a sample compound with one selected from the group consisting of
i) a ZFP91 protein,
ii) a cell line transfected with an expression vector containing a reporter gene operably linked to the downstream of response of ZFP91,
iii) a cell line transfected with an expression vector containing a reporter gene construct comprising an NFκB consensus element operably linked to the reporter gene, and
iv) a ZFP91 protein and NIK or TRAF2;
2) measuring at least one aspect selected from the group consisting of
i) binding activity between ZFP91 and the sample compound when the ZFP91 protein is contacted with the sample compound,
ii) expression of the reporter gene when the transfected cell line is contacted with the sample compound, and
iii) binding activity between the ZFP91 protein and the NIK or the TRAF2 or ubiuitinylation of the NIK or the TRAF2 when the ZFP91 protein and the NIK or the TRAF2 are contacted with the sample compound; and
3) selecting the compound enhancing at least one aspect of step 2) as compared to that in the absence of the compound.

10-13. (canceled)

14. A method for diagnosing cancer, confirming the treatment effect or evaluating prognosis comprising the step of measuring ZFP91 expression in a diagnostic sample of a subject by contacting the sample with an antibody against the ZFP91.

15-17. (canceled)

18. A method for treating a disease related to excessive angiogenesis or inflammation of a subject comprising administering a ZFP91 activation inhibitor in an amount effective to inhibit ZFP91 activation to the subject.

19. The method according to claim 18, wherein the disease is retinopathy or arthritis.

20. The method according to claim 18, wherein the disease is a chronic inflammatory disease including rheumatoid arthritis, inflammatory colitis, multiple sclerosis and chronic hepatitis.

21. (canceled)

22. An angiogenesis promoter containing a ZFP91 activation enhancer, an expression vector containing ZFP91 gene or ZFP91 protein as an active ingredient.

23. A method for treating ischemic disease in a subject, comprising administering a ZFP91 activator in an amount effective to activate ZFP91 to the subject.

24. The method according to claim 23, wherein the ischemic disease is selected from the group consisting of critical limb ischemia (CLI), coronary artery disease (CAD), dementia caused by poor blood supply, amyotrophic lateral sclerosis (ALS), diabetic neuropathy and stroke.

25. The method according to claim 23, where the ZFP91 activator promotes expression of erythropoietin (EPO).

26. The method according to claim 5, wherein the anti-angiogenic agent is an anti-cancer agent.

27. The method according to claim 5, wherein the gene regulated by ZFP91 is NIK, IKKα, p52, HIF-1α, MET, or EPO.

28. The method according to claim 6, wherein the gene regulated by ZFP91 is NIK, IKKα, p52, HIF-1α, MET, or EPO.


SEQUENCE LISTING (24 sequences)

SEQ ID NO: 1 - RNA, 15 nt, Artificial Sequence
  ZFP91 siRNA1
ggcauuaguagugaa

SEQ ID NO: 2 - RNA, 19 nt, Artificial Sequence
  antisense of ZFP91 siRNA1
uucacuacuaaugccaccu

SEQ ID NO: 3 - RNA, 19 nt, Artificial Sequence
  ZFP91 siRNA2
gcggcacacuuaucuucaa

SEQ ID NO: 4 - RNA, 19 nt, Artificial Sequence
  antisense of ZFP91 siRNA2
uugaagauaagugugccgc

SEQ ID NO: 5 - DNA, 21 bp, Artificial Sequence
  SPIN forward primer
taccacatggtctcccagttt

SEQ ID NO: 6 - DNA, 20 bp, Artificial Sequence
  SPIN reverse primer
catttgcttcaccagtgcat

SEQ ID NO: 7 - DNA, 21 bp, Artificial Sequence
  ECT2 forward primer
agctttgcaaccctgagagag

SEQ ID NO: 8 - DNA, 20 bp, Artificial Sequence
  ECT2 reverse primer
ccacaattttcccatggtct

SEQ ID NO: 9 - DNA, 20 bp, Artificial Sequence
  EWSR1 forward primer
gagctggagactggcagtgt

SEQ ID NO: 10 - DNA, 20 bp, Artificial Sequence
  EWSR1 reverse primer
tagcaccaggaagctgaggg

SEQ ID NO: 11 - DNA, 21 bp, Artificial Sequence
  MLLT4 forward primer
ctcaagcacagctgtcactca

SEQ ID NO: 12 - DNA, 20 bp, Artificial Sequence
  MLLT4 reverse primer
caagcaagcaaatccttcct

SEQ ID NO: 13 - DNA, 20 bp, Artificial Sequence
  PTPN11 forward primer
aaagaatatggcgtcatgcg

SEQ ID NO: 14 - DNA, 20 bp, Artificial Sequence
  PTPN11 reverse primer
cgtctggtccgctagagaat

SEQ ID NO: 15 - DNA, 20 bp, Artificial Sequence
  MAGEA4 forward primer
aaggagctggtcacaaaggc

SEQ ID NO: 16 - DNA, 19 bp, Artificial Sequence
  MAGEA4 reverse primer
accctgaccacatgctcca

SEQ ID NO: 17 - DNA, 21 bp, Artificial Sequence
  TNFAIP3 forward primer
gttttctggttgttgttgggg

SEQ ID NO: 18 - DNA, 21 bp, Artificial Sequence
  TNFAIP3 reverse primer
aataccagggtaccatgggat

SEQ ID NO: 19 - DNA, 21 bp, Artificial Sequence
  ZFP91 forward primer
tcagagtgttgcagatttgcc

SEQ ID NO: 20 - DNA, 21 bp, Artificial Sequence
  ZFP91 reverse primer
gggaaacggctgagatagttt

SEQ ID NO: 21 - DNA, 12 bp, Artificial Sequence
  ZFP91 promoter NF-kB binding sequence 1
acggaaattccc

SEQ ID NO: 22 - DNA, 10 bp, Artificial Sequence
  ZFP91 promoter NF-kB binding sequence 2
ggaaaaaccc

SEQ ID NO: 23 - protein, 110 aa, Artificial Sequence
  ZFP91 amino acid sequence (91-200)
VPGQQPQAAKSPSPVQGKKSPRLLCIEKVTTDKDPKEEKEEEDDSALPQEVSIAASRPSRGWRSSRTSVSRHRDTENTRSSRSKTGSLQLICKSEPNTDQLDYDVGEEHQ

SEQ ID NO: 24 - DNA, 330 bp, Artificial Sequence
  ZFP91 cDNA (encoding amino acids 91-200)
gtccccgggcagcagccccaggccgcgaagtccccgtctccagttcagggcaagaagagtccgcgactcctatgcatagaaaaagtaacaactgataaagatcccaaggaagaaaaagaggaagaagacgattctgccctccctcaggaagtttccattgctgcatctagacctagccggggctggcgtagtagtaggacatctgtttctcgccatcgtgatacagagaacacccgaagctctcggtccaagaccggttcattgcagctcatttgcaagtcagaaccaaatacagaccaacttgattatgatgttggagaagagcatcag